(19) United States
(12) Patent Application Publication
(10) Pub. No.: US 2022/0347468 A1
(43) Pub. Date: Nov. 3, 2022

(54) METHODS FOR ELECTRO-MECHANICAL TRANSFECTION

(71) Applicant: Kytopen Corporation, Cambridge, MA (US)

(72) Inventors: Paulo Andres GARCIA, Somerville, MA (US); Cullen BUIE, Cambridge, MA (US); Ross BEIGHLEY, Cambridge, MA (US); Rameech MCCORMACK, Cambridge, MA (US); James HEMPHILL, Medford, MA (US); Jessica SIDO, Alton, IL (US); Bethany GRANT, Scituate, MA (US)

(21) Appl. No.: 17/731,112

(22) Filed: Apr. 27, 2022

Related U.S. Application Data
(60) Provisional application No. 63/180,617, filed on Apr. 27, 2021.

(51) Int. Cl. A61N 1/32; C12M 1/42; C12N 13/00

(52) U.S. Cl. CPC A61N 1/327; C12M 1/42; C12M 35/02; C12N 13/00

(57) ABSTRACT

Methods, devices, systems, and kits for electro-mechanical cell transfection are provided. A device includes a first electrode, a second electrode, and an active zone therebetween where an electrical potential difference applied to the first and second electrodes generates an electric field in the active zone sufficient to transfect at least a subset of the cells flowing in the active zone.

### STATEMENT OF FEDERALLY SPONSORED RESEARCH

This invention was made with government support under Phase I SBIR Grant No. 1747096 and Phase II SBIR Grant. No 1853194 from the National Science Foundation. The government has certain rights in the invention.

### BACKGROUND OF THE INVENTION

Immunotherapy is currently at the cutting edge of both basic scientific research and pharmaceutically driven clinical application. This trend is in part due to the recent strides in targeted gene modification and the expanded use of CRISPR/Cas complex editing for therapeutic development. In order to identify genetic modifications of therapeutic interest, research organizations often have to screen thousands of genetic variants, which can include modification of an endogenous gene or insertion of an engineered gene. This drug discovery process is laborious, typically requiring significant manual labor within the laboratory, creating an industry-wide bottleneck due to the lack of adequate high-throughput technologies.

Biotech and pharmaceutical research and development activities have shifted to automating nearly all steps of the process. The workflows include liquid handling robots, powered by sophisticated laboratory management software, to enable high throughput discovery. However, transfection steps are limited to low throughput, poor efficiency technologies, and user-intensive systems that cannot be automated. Automated platforms for transfection not only have the potential to reduce process costs substantially, but also increase cell viability and the quantity of successfully engineered cells, all while reducing discovery time, which is critical in the competitive immunotherapy space.

A unique strength of transfection by way of electroporation is RNA delivery. Existing viral techniques to deliver DNA appear on par with transfection by way of electroporation, but there is a lack of GMP-quality non-retroviral RNA viruses. Therefore, companies with electroporation platforms have been the target of collaborations and acquisitions for the purpose of delivering mRNA into cells.

Current high-throughput gene transfer methods typically require the use of viral delivery (e.g., lentiviral vectors), in which viral particles infect a cell and transduce the genetic modification of interest. While a viral methodology can be applied to high-throughput automated systems, there are limitations in the production that extend timelines for research efforts: viral vectors have to be cloned, transfected into a viral production line, and then viral particles must be purified. This process can take research organizations months, significantly affecting their timelines for platform development while simultaneously increasing the cost of drug discovery. Additionally, the use of viral transduction for gene transfer is not amenable to genetic modification for all cell types, since some cells (such as specific immune cell subsets) are resistant to viral infection. Therefore, within the biotechnology industry there is an unmet need to have a high-throughput automated system for gene transfer that does not rely on viral delivery mechanisms.

### SUMMARY OF THE INVENTION

We demonstrate through the work described herein that a non-viral approach employing electro-mechanical transfection involving the use of electric fields coupled with high fluid flow rates is a scalable strategy for the development and manufacturing of ex vivo cell therapies. Unlike either purely electric field-based or purely mechanical-based poration methods, the combined effects of electro-mechanical transfection result in delivery of genetic material at high efficiencies and low toxicity. The invention shows for the first time that electro-mechanical transfection can be used successfully in human primary T cells.

Utilizing a commercially available liquid handling system, rapid optimization of delivery to expanded T cell was observed with efficiency greater than 90% and greater than 80% viability. Confirmation of optimized electro-mechanical transfection parameters was assessed in multiple use cases including delivery to naïve T cells, NK cells, B cells, monocytes, macrophages, delivery of multiple payloads, and in a 50-fold scale up demonstration. Additionally, transcriptome and ontology analysis show that high efficiency, high viability delivery, via electro-mechanical transfection results in minimal gene dysregulation (only 2% change from baseline). The invention demonstrates that non-viral electro-mechanical transfection is an efficient and scalable method for cell and gene therapy engineering and development.

Accordingly, in one aspect, the invention provides a method of introducing a composition into a plurality of mammalian cells suspended in a flowing liquid using any of the devices or systems of the invention (e.g., by electro-mechanical transfection). In particular, methods of the invention include providing a device including an entry zone comprising a first inlet and a first outlet; first and second electrodes; and an active zone comprising a second inlet and second outlet. The method further includes selecting a combination of an electric field (E), an average flow velocity (u), a hydraulic diameter (d) in the active zone, a liquid conductivity (σ), liquid dynamic viscosity (μ), and liquid density (ρ) to give a dimensionless parameter π₅ having a value of between 1×10⁸ and 1×10¹⁰; where the dimensionless parameter π₅ is represented by

$${Π}_{5} = \frac{ρσ {d}^{3}}{{μ}^{2}} ( \frac{{E}^{2}}{u} ) .$$

The method further includes passing the plurality of cells and the composition through the active zone while providing the selected combination of (E), (u), (d), (σ), (μ), and (ρ), thereby introducing the composition into the plurality of mammalian cells.

In some embodiments, the composition is introduced into the plurality of cells at a flux of at least 1×10⁵ cells per minute per active zone, e.g., 10⁵ cells/min to 10¹² cells/min, e.g., 10⁵ cells/min to 10⁶ cells/min, 5×10⁵ cells/min to 5×10⁶ cells/min, 10⁶ cells/min to 10⁷ cells/min, 5×10⁶ cells/min to 5×10⁷ cells/min, 10⁷ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, or 10¹⁰ cells/min to 10¹¹ cells/min, e.g., about 10³ cells/min, 5×10³ cells/min, 10⁴ cells/min, 5×10⁴ cells/min, 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹¹ cells/min, or 10¹¹ cells/min per active zone.

In some embodiments, the entry zone and active zone are configured to provide an increase in average flow velocity, e.g., relative to an average flow velocity in the entry zone.

In another aspect, the invention provides a method of introducing a composition into a plurality of cells suspended in a flowing liquid. The method includes providing a device including an entry zone with a first inlet and a first outlet, first and second electrodes, and an active zone including a second inlet and second outlet. The method further includes passing the plurality of cells and composition through the active zone while providing electrical energy from the first and second electrodes and simultaneously providing mechanical energy at least partially from an average flow velocity, where the mechanical energy and the electrical energy together result in introducing the composition into the plurality of cells with an efficiency, a yield, and/or a viability at least equal to electroporation or mechanical poration alone with less electrical or mechanical energy than electroporation of mechanical poration require to achieve the efficiency, yield, and/or viability.

In some embodiments, the composition is introduced with a flux of at least 1×10⁵ cell/min per active zone, e.g., 10⁵ cells/min to 10⁶ cells/min, 5×10⁵ cells/min to 5×10⁶ cells/min, 10⁶ cells/min to 10⁷ cells/min, 5>10⁶ cells/min to 5×10⁷ cells/min, 10′ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, or 10¹⁰ cells/min to 10¹¹ cells/min, e.g., about 10³ cells/min, 5×10³ cells/min, 10⁴ cells/min, 5×10⁴ cells/min, 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹⁰ cells/min, 10¹¹ cells/min, 5×10¹¹ cells/min, or 10¹² cells/min, per active zone.

In some embodiments, the ratio of electrical energy provided by the electric field to the mechanical energy provided to the flowing liquid by a product of a pressure drop and the flow rate in the active zone is between 10³:1 and 10⁶:1, e.g., between 10³:1 and 10⁵:1, 10⁴:1 and 10⁶:1, 10³:1 and 10⁴:1, or 10⁵:1 and 10⁶:1 (e.g., about 10³:1, 10⁴:1, 10⁵:1, or 10⁶:1). In some embodiments, the entry zone and active zone are configured to provide an increase in average flow velocity.

Another aspect of the invention provides a method of introducing a composition into a plurality of cells suspended in a flowing liquid. The method includes providing a device including an entry zone with a first inlet and a first outlet, first and second electrodes, and an active zone including a second inlet and second outlet, and having a hydraulic diameter (d). The method further includes providing a test portion of the plurality of cells and a test composition having together a liquid conductivity (σ), liquid dynamic viscosity (μ), and liquid density (ρ), and a ratio of cells to composition, and passing the test portion and test composition through the active zone at an average flow velocity (u) while applying an electric field (E), where at least one of (u), (E), (σ), (μ), and (ρ) is varied. The method further includes identifying a range of a dimensionless parameter π₅ which includes a maximum yield, efficiency, and/or cell viability for introduction of the test composition into the test portion of the plurality of cells, where

$${Π}_{5} = \frac{ρσ {d}^{3}}{{μ}^{2}} ( \frac{{E}^{2}}{u} ) .$$

The plurality of cells and the composition are then passed through the active zone with combination of (u), (E), (σ), (μ), and (ρ) corresponding to a value of π₅ which includes at least one of the maximum yield, efficiency, or cell viability, thereby introducing the composition into the plurality of cells.

In some embodiments, the range identification is repeated where the test portion of the plurality of cells and test composition have a second ratio of cells to composition, and/or with the active zone having a second hydraulic diameter (d).

In some embodiments, the method includes varying the average velocity (u) while holding the electric field (E) constant or varying the electric field (E) while the average flow velocity (u) is held constant during the π₅ range identification step.

In some embodiments, the composition is introduced with a flux of at least 1×10⁵ cells per minute per active zone, e.g., 10⁵ cells/min to 10¹² cells/min per active zone, e.g., 10⁵ cells/min to 10⁶ cells/min, 5×10⁵ cells/min to 5×10⁶ cells/min, 10⁶ cells/min to 10⁷ cells/min, 5×10⁶ cells/min to 5×10′ cells/min, 10⁷ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, or 10¹⁰ cells/min to 10¹¹ cells/min per active zone, e.g., about 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹⁰ cells/min, 10¹¹ cells/min, 5×10¹¹ cells/min, or 10¹² cells/min per active zone.

In some embodiments, the entry zone and active zone are configured to provide an increase in average flow velocity (u).

In another aspect, the invention provides a method of introducing a composition into a plurality of human immune cells isolated from a suspension collected from normal patient or donor blood and suspended in a flowing liquid. The method includes providing a device including an entry zone comprising a first inlet and a first outlet, first and second electrodes, and an active zone comprising a second inlet and second outlet. The method further includes selecting a combination of an electric field (E), an average flow velocity (u), a hydraulic diameter (d) in the active zone, a liquid conductivity (a), liquid dynamic viscosity (μ) (e.g., as measured by rotational viscometry), and liquid density (p) to give a dimensionless parameter π₅ having a value of between 1×10⁸ and 1×10¹⁰; where the dimensionless parameter π₅ is represented by

$${Π}_{5} = \frac{ρσ {d}^{3}}{{μ}^{2}} ( \frac{{E}^{2}}{u} ) .$$

The method further includes passing the plurality of cells and the composition through the active zone while providing the selected combination of (E), (u), (d), (σ), (μ), and (ρ), thereby introducing the composition into the plurality of human immune cells to produce a therapeutic dose of transfected cells.

In some embodiments, the suspension is prepared using leukapheresis. In some embodiments, the composition is introduced into the plurality of cells at a flux of at least 1×10⁵ cells per minute per active zone, e.g., 10⁵ cells/min to 10¹² cells/min per active zone, e.g., 10⁵ cells/min to 10⁶ cells/min, 5×10⁵ cells/min to 5×10⁶ cells/min, 10⁶ cells/min to 10⁷ cells/min, 5×10⁶ cells/min to 5×10⁷ cells/min, 10⁷ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, or 10¹⁰ cells/min to 10¹¹ cells/min per active zone, e.g., about 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹⁰ cells/min, 10¹¹ cells/min, 5×10¹¹ cells/min, or 10¹² cells/min per active zone. In some embodiments, the entry zone and active zone are configured to provide an increase in average flow velocity, e.g., relative to a flow velocity through the entry zone. In some embodiments, the composition is introduced into the plurality of mammalian cells without altering a desired cell surface marker. Cell surface markers include, but are not limited to, CD3, CD4, CD8, CD19, CD45RA, CD45RO, CD28, CD44, CD69, CD80, CD86, CD206, IL-2 receptor, CTLA4, OX40, PD-1, TIM3, CD56, TNFa, IFNg, LAG3, TCR alpha/beta, CD64, SIRP alpha/beta (CD172a/b), Nestin, CD325 (N-Cadherin), CD183 (CXCR3), CD184 (CXCR4), CD197 (CCR7), CD27, CD11b, CCR7 (CD197), CD16, CD56, TIGIT, TRA-1-60, Nanog, TCR gamma/delta, OCT4, T-bet, GATA-3, FoxP3, IL-17, B220, CD25, IgM, PD-L1, IL-23, IL-12, CD11c, and F4/80.

In some embodiments of any of the preceding aspects, the active zone includes a minimum hydraulic diameter greater than 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, 1.0 mm, 1.5 mm, 2 mm, 5 mm, 10 mm, 15 mm, 25 mm, or 50 mm). In some embodiments, any of the preceding aspects, the active zone includes a minimum hydraulic diameter greater than an average cell diameter in the plurality of cells, e.g., at least 1.1 times the average cell diameter, e.g., between 1 and 10 times (e.g., 1-2 time, 2-3 time, 3-4 times, 4-5 times, 5-6 times, 6-7 times, 7-8 times, 8-9 times, or 1-10 times) the average cell diameter, or, e.g., between 10 and 100 times (e.g., 10-20 time, 20-30 time, 30-40 times, 40-50 times, 50-60 times, 60-70 times, 70-80 times, 80-90 times, or 10-100 times) the average cell diameter, or, e.g., between 100 and 1,000 times (e.g., 100-200 time, 200-300 time, 300-400 times, 400-500 times, 500-600 times, 600-700 times, 700-800 times, 800-900 times, or 100-1,000 times) the average cell diameter, or, e.g., between 1,000 and 10,000 times (e.g., 1,000-2,000 time, 2,000-3,000 time, 3,000-4,000 times, 4,000-5,000 times, 5,000-6,000 times, 6,000-7,000 times, 7,000-8,000 times, 8,000-9,000 times, or 1,000-10,000 times) the average cell diameter, or, e.g., greater than 10,000 times the average cell diameter, e.g. 12,000 time, 15,000 time, 18, 000 times or 20,000 times the average cell diameter.

In some embodiments of any of the preceding aspects, the active zone has a substantially uniform cross-sectional area.

In some embodiments of any of the preceding aspects, the flow rate through the active zone is between 0.001 mL/min and 1,000 mL/min (e.g., between 0.001 mL/min and 0.05 mL/min, between 0.001 mL/min and 0.1 mL/min, between 0.001 mL/min and 1 mL/min, between 0.05 mL/min and 0.5 mL/min, between 0.05 mL/min and 5 mL/min, between 0.1 mL/min and 1 mL/min, between 0.5 mL/min and 2 mL/min, between 1 mL/min and 5 mL/min, between 1 mL/min and 10 mL/min, between 1 mL/min and 100 mL/min, between 5 mL/min and 25 mL/min, between 5 mL/min and 150 mL/min, between 10 mL/min and 100 mL/min, between 15 mL/min and 150 mL/min, between 25 mL/min and 100 mL/min, between 25 mL/min and 200 mL/min, between 50 mL/min and 150 mL/min, between 50 mL/min and 250 mL/min, between 75 mL/min and 200 mL/min, between 75 mL/min and 350 mL/min, between 100 mL/min and 250 mL/min, between 100 mL/min and 400 mL/min, between 150 mL/min and 450 mL/min, between 200 mL/min and 500 mL/min, between 250 mL/min and 700 mL/min, between 300 mL/min and 1,000 mL/min, between 400 mL/min and 750 mL/min, between 500 mL/min and 1,000 mL/min, or between 750 mL/min and 1,000 mL/min, e.g., about 0.001 mL/min, 0.01 mL/min, 0.05 mL/min, 0.1 mL/min, 0.5 mL/min, 1 mL/min, 5 mL/min, 10 mL/min, 15 mL/min, 20 mL/min, 30 mL/min, 40 mL/min, 50 mL/min, 60 mL/min, 70 mL/min, 80 mL/min, 90 mL/min, 100 mL/min, 150 mL/min, 200 mL/min, 250 mL/min, 300 mL/min, 350 mL/min, 400 mL/min, 450 mL/min, 500 mL/min, 600 mL/min, 700 mL/min, 800 mL/min, 900 mL/min, or 1,000 mL/min).

In some embodiments of any of the preceding aspects, a Reynolds number (ρud/μ) (based on the hydraulic diameter (d) of the active zone) of the flowing liquid in active zone is between 10 and 3000 (e.g., between 10 and 2000, between 100 and 1600, between 100 and 1800, or between 183 and 1530).

In some embodiments, the average flow velocity of the flowing liquid in suspension therein flowing through active zone is between 1×10⁻² m/s and 10 m/s, e.g., between 0.01 and 1 m/s (e.g., between 0.01 and 0.05 m/s, 0.05 and 0.1 m/s, 0.1 and 0.5 m/s, 0.5 and 1 m/s, 1.5 and 2 m/s, 1 and 2 m/s, 2 and 3 m/s, 3 and 4 m/s, 4 and 5 m/s, 5 and 6 m/s, 6 and 7 m/s, 7 and 8 m/s, 8 and 9 m/s, or 9 and 10 m/s), e.g., between 0.1 and 5 m/s, between 0.4 and 1.4 m/s, between 0.65 and 1.3 m/s, or between 0.26 and 2.08 m/s, e.g., about 0.1 m/s, 0.2 m/s, 0.3 m/s, 0.4 m/s, 0.5 m/s, 0.6 m/s, 0.7 m/s, 0.8 m/s, 0.9 m/s, 1.0 m/s, 1.1 m/s, 1.2 m/s, 1.3 m/s, 1.4 m/s, 1.5 m/s, 2 m/s, 3 m/s, 4 m/s, 5 m/s, 6 m/s, 7 m/s, 8 m/s, 9 m/s, or 10 m/s.

In some embodiments, a peak pressure of the flowing liquid in suspension while passing through the active zone is between 1×10⁻³ Pa and 9.5×10⁴ Pa (e.g., between 0.1 Pa and 10,000 Pa, between 1 Pa and 5,000 Pa, between 100 Pa and 3000 Pa, or between 136 Pa and 1600 Pa).

In some embodiments of any of the previous aspects, a residence time in the active zone of any of the plurality of cells suspended in the liquid is between 0.1 ms and 50 ms (e.g., between 0.1 and 0.5 ms, between 0.5 ms and 5 ms, between 1 ms and 10 ms, between 1 ms and 15 ms, between 5 ms and 15 ms, between 10 ms and 20 ms, between 15 ms and 25 ms, between 20 ms and 30 ms, between 25 ms and 35 ms, between 30 ms and 40 ms, between 35 ms and 45 ms, or between 40 ms and 50 ms, e.g., about 0.5 ms, 0.6 ms, 0.7 ms, 0.8 ms, 0.9 ms, 1 ms, 1.5 ms, 2 ms, 2.5 ms, 3 ms, 3.5 ms, 4 ms, 4.5 ms, 5 ms, 5.5 ms, 6 ms, 6.5 ms, 7 ms, 7.5 ms, 8 ms, 8.5 ms, 9 ms, 9.5 ms, 10 ms, 10.5 ms, 11 ms, 11.5 ms, 12 ms, 12.5 ms, 13 ms, 13.5 ms, 14 ms, 14.5 ms, 15 ms, 20 ms, 25 ms, 30 ms, 35 ms, 40 ms, 45 ms, or 50 ms). In some embodiments, the residence time is from 5-20 ms (e.g., from 6-18 ms, 8-15 ms, or 10-14 ms).

In some embodiments of any of the preceding aspects, the electric field is produced by voltage pulses, wherein the voltage pulses energize the first electrode at a particular applied voltage while the second electrode is energized at a particular applied voltage, thus applying an electrical potential difference between the first and second electrodes, wherein the voltage pulses each have an amplitude between −3 kV and 3 kV (e.g., between −3 kV and 1 kV, between −3 kV and −1.5 kV, between −2 kV and 2 kV, between −1.5 kV and 1.5 kV, between −1.5 kV and 2.5 kV, between −1 kV and 1 kV, between −1 kV and 2 kV, between −0.5 kV and 0.5 kV, between −0.5 kV and 1.5 kV, between −0.5 kV and 3 kV, between −0.01 kV and 2 kV, between 0 kV and 1 kV, between 0 kV and 2 kV, between 0 kV and 3 kV, between 0.01 kV and 0.1 kV, between 0.01 kV and 1 kV, between 0.02 kV and 0.2 kV, between 0.03 kV and 0.3 kV, between 0.04 kV and 0.4 kV, between 0.05 kV and 0.5 kV, between 0.05 kV and 1.5 kV, between 0.06 kV and 0.6 kV, between 0.07 kV and 0.7 kV, between 0.08 kV and 0.8 kV, between 0.09 kV and 0.9 kV, between 0.1 kV and 0.7 kV, between 0.1 kV and 1 kV, between 0.1 kV and 2 kV, between 0.1 kV and 3 kV, between 0.15 kV and 1.5 kV, between 0.2 and 0.6 kV, between 0.2 kV and 2 kV, between 0.25 kV and 2.5 kV, between 0.3 kV and 3 kV, between 0.5 kV and 1 kV, between 0.5 kV and 3 kV, between 0.6 kV and 1.5 kV, between 0.7 kV and 1.8 kV, between 0.8 kV and 2 kV, between 0.9 kV and 3 kV, between 1 kV and 2 kV, between 1.5 kV and 2.5 kV, or between 2 kV and 3 kV, e.g., about −3 kV, −2.5 kV, −2 kV, −1.5 kV, −1 kV, −0.5 kV, −0.01 kV, 0 kV, 0.01 kV, 0.02 kV, 0.03 kV, 0.04 kV, 0.05 kV, 0.06 kV, 0.07 kV, 0.08 kV, 0.09 kV, 0.1 kV, 0.2 kV, 0.3 kV, 0.4 kV, 0.5 kV, 0.6 kV, 0.7 kV, 0.8 kV, 0.9 kV, 1 kV, 1.1 kV, 1.2 kV, 1.3 kV, 1.4 kV, 1.5 kV, 1.6 kV, 1.7 kV, 1.8 kV, 1.9 kV, 2 kV, 2.1 kV, 2.2 kV, 2.3 kV, 2.4 kV, 2.5 kV, 2.6 kV, 2.7 kV, 2.8 kV, 2.9 kV, or 3 kV). In some embodiments, the first electrode is energized at a particular applied voltage while the second electrode is held at ground (e.g., 0 kV), thus applying an electrical potential difference between the first and second electrodes. In some embodiments, the voltage pulses have a duration of between 0.01 ms and 1,000 ms (e.g., between 0.01 ms and 0.1 ms, between 0.01 ms and 1 ms, between 0.01 ms and 10 ms, between 0.05 ms and 0.5 ms, between 0.05 ms and 1 ms, between 0.1 ms and 1 ms, between 0.1 ms and 5 ms, between 0.1 ms and 500 ms, between 0.5 ms and 2 ms, between 1 ms and 5 ms, between 1 ms and 10 ms, between 1 ms and 25 ms, between 1 ms and 100 ms, between 1 ms and 1,000 ms, between 5 ms and 25 ms, between 5 ms and 150 ms, between 10 ms and 100 ms, between 15 ms and 150 ms, between 25 ms and 100 ms, between 25 ms and 200 ms, between 50 ms and 150 ms, between 50 ms and 250 ms, between 75 ms and 200 ms, between 75 ms and 350 ms, between 100 ms and 250 ms, between 100 ms and 400 ms, between 150 ms and 450 ms, between 200 ms and 500 ms, between 250 ms and 700 ms, between 300 ms and 1,000 ms, between 400 ms and 750 ms, between 500 ms and 1,000 ms, or between 750 ms and 1,000 ms, e.g., about 0.01 ms, 0.05 ms, 0.1 ms, 0.5 ms, 1 ms, 5 ms, 10 ms, 15 ms, 20 ms, 30 ms, 40 ms, 50 ms, 60 ms, 70 ms, 80 ms, 90 ms, 100 ms, 150 ms, 200 ms, 250 ms, 300 ms, 350 ms, 400 ms, 450 ms, 500 ms, 600 ms, 700 ms, 800 ms, 900 ms, or 1,000 ms). In some embodiments, the voltage pulses have a duration of at least 1000 ms. In some embodiments, the voltage pulses are applied to the first and second electrodes at a frequency of between 1 Hz and 50,000 Hz (e.g., between 1 Hz and 10 Hz, between 1 Hz and 100 Hz, between 1 Hz and 1,000 Hz, between 5 Hz and 20 Hz, between 5 Hz and 2,000 Hz, between 10 Hz and 50 Hz, between 10 Hz and 100 Hz, between 10 Hz and 1,000 Hz, between 10 Hz and 10,000 Hz, between 20 Hz and 50 Hz, between 20 Hz and 100 Hz, between 20 Hz and 2,000 Hz, between 20 Hz and 20,000 Hz, between 50 Hz and 500 Hz, between 50 Hz and 1,000 Hz, between 50 Hz and 50,000 Hz, between 100 Hz and 200 Hz, between 100 Hz and 500 Hz, between 100 Hz and 1,000 Hz, between 100 Hz and 10,000 Hz, between 100 Hz and 50,000 Hz, between 200 Hz and 400 Hz, between 200 Hz and 750 Hz, between 200 Hz and 2,000 Hz, between 500 Hz and 1,000 Hz, between 750 Hz and 1,500 Hz, between 750 Hz and 10,000 Hz, between 1,000 Hz and 2,000 Hz, between 1,000 Hz and 5,000 Hz, between 1,000 Hz and 10,000 Hz, between 1,000 Hz and 50,000 Hz, between 5,000 Hz and 10,000 Hz, between 5,000 Hz and 20,000 Hz, between 5,000 Hz and 50,000 Hz, between 10,000 Hz and 15,000 Hz, between 10,000 Hz and 25,000 Hz, between 10,000 Hz and 50,000 Hz, between 20,000 Hz and 30,000 Hz, or between 20,000 and 50,000 Hz, e.g., about 1 Hz, 5 Hz, 10 Hz, 20 Hz, 50 Hz, 75 Hz, 100 Hz, 150 Hz, 200 Hz, 300 Hz, 400 Hz, 500 Hz, 600 Hz, 700 Hz, 800 Hz, 900 Hz, 1,000 Hz, 2,000 Hz, 5,000 Hz, 10,000 Hz, 15,000 Hz, 20,000 Hz, 30,000 Hz, 40,000 Hz, or 50,000 Hz).

In some embodiments, a waveform of the voltage pulse is selected from a group consisting of DC, square, pulse, bipolar, sine, ramp, asymmetric bipolar, arbitrary, and any superposition or combinations thereof. In some embodiments, the electric field generated from the voltage pulses has a magnitude of between 100 V/cm and 50,000 V/cm (e.g., between 100 V/cm and 500 V/cm, between 100 V/cm and 1,000 V/cm, between 100 V/cm and 2,000 V/cm, between 100 V/cm and 5,000 V/cm, between 250 V/cm and 2000 V/cm, between 500 V/cm and 2500 V/cm, between 500 V/cm and 5,000 V/cm, between 500 V/cm and 1,500 V/cm, between 300 V/cm and 500 V/cm, between 1000 V/cm and 2,000 V/cm, e.g., about 100 V/cm, 150 V/cm, 200 V/cm, 250 V/cm, 300 V/cm, 350 V/cm, 400 V/cm, 450 V/cm, 500 V/cm, 550 V/cm, 600 V/cm, 650 V/cm, 700 V/cm, 750 V/cm, 800 V/cm, 900 V/cm, 1,000 V/cm, or 2,000 V/cm).

In some embodiments, a duty cycle of the voltage pulses is between 1% and 100% (e.g., between 1% and 10%, between 1% and 97%, between 2.5% and 20%, between 5% and 25%, between 5% and 40%, between 10% and 25%, between 10% and 50%, between 10% and 95%, between 15% and 60%, between 15% and 85%, between 20% and 40%, between 30% and 50%, between 40% and 60%, between 40% and 75%, between 50% and 85%, between 50% and 100%, between 75% and 100%, or between 90% and 100%, e.g., about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%).

In some embodiments, the liquid has a conductivity of between 0.001 mS/cm and 500 mS/cm (e.g., between 0.001 mS/cm and 0.05 mS/cm, between 0.001 mS/cm and 0.1 mS/cm, between 0.001 mS/cm and 1 mS/cm, between 0.05 mS/cm and 0.5 mS/cm, between 0.05 mS/cm and 5 mS/cm, between 0.1 mS/cm and 1 mS/cm, between 0.1 mS/cm and 100 mS/cm, between 0.5 mS/cm and 2 mS/cm, between 1 mS/cm and 5 mS/cm, between 1 mS/cm and 10 mS/cm, between 1 mS/cm and 100 mS/cm, between 1 mS/cm and 500 mS/cm, between 5 mS/cm and 25 mS/cm, between 5 mS/cm and 150 mS/cm, between 10 mS/cm and 100 mS/cm, between 10 mS/cm and 250 mS/cm, between 15 mS/cm and 150 mS/cm, between 25 mS/cm and 100 mS/cm, between 25 mS/cm and 200 mS/cm, between 50 mS/cm and 150 mS/cm, between 50 mS/cm and 250 mS/cm, between 50 mS/cm and 500 mS/cm, between 75 mS/cm and 200 mS/cm, between 75 mS/cm and 350 mS/cm, between 100 mS/cm and 250 mS/cm, between 100 mS/cm and 400 mS/cm, between 100 mS/cm and 500 mS/cm, between 150 mS/cm and 450 mS/cm, between 200 mS/cm and 500 mS/cm, between 300 mS/cm and 500 mS/cm, e.g., about 0.001 mS/cm, 0.01 mS/cm, 0.05 mS/cm, 0.1 mS/cm, 0.5 mS/cm, 1 mS/cm, 5 mS/cm, 10 mS/cm, 15 mS/cm, 20 mS/cm, 30 mS/cm, 40 mS/cm, 50 mS/cm, 60 mS/cm, 70 mS/cm, 80 mS/cm, 90 mS/cm, 100 mS/cm, 150 mS/cm, 200 mS/cm, 250 mS/cm, 300 mS/cm, 350 mS/cm, 400 mS/cm, 450 mS/cm, or 500 mS/cm).

In some embodiments, a temperature of the plurality of cells suspended in the liquid is between 0° C. and 50° C. (between 0° C. and 5° C., between 2° C. and 15° C., between 3° C. and 30° C., between 4° C. and 10° C., between 4° C. and 25° C., between 5° C. and 30° C., between 7° C. and 35° C., between 10° C. and 25° C., between 10° C. and 40° C., between 15° C. and 50° C., between 20° C. and 40° C., between 25° and 50° C., or between 35° C. and 45° C., e.g., about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C. 22° C., 23° C., 24° C., 25° C., 26° C., 27° C., 28° C., 29° C., 30° C. 31° C., 32° C., 33° C., 34° C., 35° C., 36° C., 37° C., 38° C., 39° C., or 40° C.).

In some embodiments, the method further includes storing the plurality of cells suspended in the liquid in a recovery buffer after transfection. In some embodiments, the cells have a viability after introduction of the composition of between 0.1% and 99.9% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 99.9%, between 60% and 80%, between 75% and 99.9%, or between 85% and 99.9%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 99.9%).

In some embodiments, the composition is introduced into a plurality of the cells at an efficiency of between 0.1% and 99.9% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 99.9%, between 60% and 80%, between 75% and 99.9%, or between 85% and 99.9%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 99.9%).

In some embodiments, the method produces a cell recovery rate of between 0.1% and 100% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 75% and 100%, between 85% and 100%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%). In some embodiments, the method produces a live engineered cell yield (e.g., a recovery yield) of between 0.1% and 500% at 24 hours post-transfection (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 60% and 150%, between 75% and 100%, between 75% and 200%, between 85% and 150%, between 90% and 250%, between 100% and 200%, between 100% and 400%, between 150% and 300%, between 200% and 500%, or between 300% and 500%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 100%, 150%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, or 500%).

In some embodiments, the method produces a live engineered cell yield (e.g., a recovery yield) of between 0.1% and 100% immediately after transfection (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 60% and 90%, between 75% and 100%, or between 85% and 100%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100).

In some embodiments, the composition delivered to the plurality of cells (e.g., electro-mechanically delivered into the plurality of cells) includes at least one compound selected from the group consisting of therapeutic agents, vitamins, nanoparticles, charged molecules, uncharged molecules, engineered nucleases, DNA, RNA, CRISPR-Cas complex, transcription activator-like effector nucleases (TALENs), zinc-finger nucleases (ZFNs), homing nucleases, meganucleases (mns), megaTALs, enzymes, transposons, peptides, proteins, viruses, polymers, a ribonucleoprotein (RNP), and polysaccharides. In some embodiments, the composition has a concentration in the liquid of between 0.0001 μM and 20 μM (e.g., from 0.0001 μM to 0.001 μM, 0.001 μM to 0.01 μM, 0.001 μM to 5 μM, 0.005 μM to 0.1 μM, 0.01 μM to 0.1 μM, 0.01 μM to 1 μM, 0.1 μM to 1 μM, 0.1 μM to 5 μM, 1 μM to 10 μM, 1 μM to 15 μM, or 1 μM to 20 μM, e.g., about 0.0001 μM, 0.0005 μM, 0.001 μM, 0.005 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 11 μM, 12 μM, 13 μM, 14 μM, 15 μM, 16 μM, 17 μM, 18 μM, 19 μM, or 20 μM). In some embodiments, the composition has a concentration in the liquid of between 0.0001 μg/mL and 1,000 μg/mL (e.g., from 0.0001 μg/mL to 0.001 μg/mL, 0.001 μg/mL to 0.01 μg/mL, 0.001 μg/mL to 5 μg/mL, 0.005 μg/mL to 0.1 μg/mL, 0.01 μg/mL to 0.1 μg/mL, 0.01 μg/mL to 1 μg/mL, 0.1 μg/mL to 1 μg/mL, 0.1 μg/mL to 5 μg/mL, 1 μg/mL to 10 μg/mL, 1 μg/mL to 50 μg/mL, 1 μg/mL to 100 μg/mL, 2.5 μg/mL to 15 μg/mL, 5 μg/mL to 25 μg/mL, 5 μg/mL to 50 μg/mL, 5 μg/mL to 500 μg/mL, 7.5 μg/mL to 75 μg/mL, 10 μg/mL to 100 μg/mL, 10 μg/mL to 1,000 μg/mL, 25 μg/mL to 50 μg/mL, 25 μg/mL to 250 μg/mL, 25 μg/mL to 500 μg/mL, 50 μg/mL to 100 μg/mL, 50 μg/mL to 250 μg/mL, 50 μg/mL to 750 μg/mL, 100 μg/mL to 300 μg/mL, 100 μg/mL to 1,000 μg/mL, 200 μg/mL to 400 μg/mL, 250 μg/mL to 500 μg/mL, 350 μg/mL to 500 μg/mL, 400 μg/mL to 1,000 μg/mL, 500 μg/mL to 750 μg/mL, 650 μg/mL to 1,000 μg/mL, or 800 μg/mL to 1,000 μg/mL, e.g., about 0.0001 μg/mL, 0.0005 μg/mL, 0.001 μg/mL, 0.005 μg/mL, 0.01 μg/mL, 0.02 μg/mL, 0.03 μg/mL, 0.04 μg/mL, 0.05 μg/mL, 0.06 μg/mL, 0.07 μg/mL, 0.08 μg/mL, 0.09 μg/mL, 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2 μg/mL, 2.5 μg/mL, 3 μg/mL, 3.5 μg/mL, 4 μg/mL, 4.5 μg/mL, 5 μg/mL, 5.5 μg/mL, 6 μg/mL, 6.5 μg/mL, 7 μg/mL, 7.5 μg/mL, 8 μg/mL, 8.5 μg/mL, 9 μg/mL, 9.5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, or 1,000 μg/mL).

In some embodiments of any of the preceding aspects, the plurality of cells suspended in the liquid includes eukaryotic cells (e.g., animal cells, e.g., human cells), prokaryotic cells (e.g., bacterial cells), plant cells, and/or synthetic cells. The cells can be primary cells (e.g., primary human cells), cells from a cell line (e.g., a human cell line), cells in suspension, adherent cells, stem cells, blood cells (e.g., peripheral blood mononuclear cells (PBMCs)), and/or immune cells (e.g., white blood cells (e.g., innate immune cells or adaptive immune cells)). In some embodiments, the cells (e.g., immune cells, e.g., T cells, B cell, natural killer cells, macrophages, monocytes, or antigen-presenting cells) are unstimulated cells, stimulated cells, or activated cells. In some embodiments, the cells are adaptive immune cells and/or innate immune cells. In some embodiments, the plurality of cells includes antigen presenting cells (APCs), monocytes, T-cells, B-cells, dendritic cells, macrophages, neutrophils, NK cells, Jurkat cells, THP-1 cells, human embryonic kidney (HEK-293) cells, Chinese hamster ovary (e.g., CHO-K1) cells, embryonic stem cells (ESCs), mesenchymal stem cells (MSCs), or hematopoietic stem cells (HSCs). In some embodiments, the cells can be primary human T-cells, primary human macrophages, primary human monocytes, primary human NK cells, or primary human induced pluripotent stem cells (iPSCs). In some embodiments of any of the methods described herein, the method further includes storing the plurality of cells suspended in the liquid in a recovery buffer after poration.

In some embodiments of any of the preceding aspects, the invention provides a kit including any of the devices or systems described herein; and, e.g., a plurality of outer structures configured to encase the plurality of devices, wherein each of the plurality of outer structures includes: a housing configured to encase the first electrode, second electrode, and the active zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase a temperature of the at least one device, wherein the thermal controller is a heating element selected from a group consisting of a heating block, a liquid flow, a battery-powered heater, and a thin-film heater. In some embodiments, the housing further includes a thermal controller configured to decrease a temperature of the at least one device, wherein the thermal controller is a cooling element selected from a group consisting of a liquid flow, an evaporative cooler, and a Peltier device.

In some embodiments of any of the preceding aspects, the invention provides a kit for introducing a composition into a plurality of cells suspended in a liquid, wherein the kit includes a plurality of devices described herein and a plurality of outer structures configured to encase the plurality of devices, wherein each of the plurality of outer structures includes: a housing configured to encase the first electrode, second electrode, and the active zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase a temperature of the at least one device, wherein the thermal controller is a heating element selected from a group consisting of a heating block, a liquid flow, a battery-powered heater, and a thin-film heater. In some embodiments, the housing further includes a thermal controller configured to decrease a temperature of the at least one device, wherein the thermal controller is a cooling element selected from a group consisting of a liquid flow, an evaporative cooler, and a Peltier device.

In some embodiments of any of the preceding aspects, the invention provides a kit for electro-mechanically delivering a composition into a plurality of cells suspended in a liquid, including: a plurality of devices, each of the plurality of devices including a device of the aforementioned embodiments; and a plurality of outer structures configured to encase the plurality of devices, wherein each of the plurality of outer structures includes: a housing configured to encase the first electrode, second electrode, and the active zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase the temperature of the at least one device, wherein the thermal controller is a heating element selected from a group consisting of a heating block, a liquid flow, a battery-powered heater, and a thin-film heater. In some embodiments, the housing further includes a thermal controller configured to decrease the temperature of the at least one device, wherein the thermal controller is a cooling element selected from a group consisting of a liquid flow, an evaporative cooler, and a Peltier device.

In some embodiments of any of the preceding methods, the kit further includes one or more reservoirs, e.g., a first reservoir and a second reservoir, fluidically connected to a zone, e.g., the entry zone, active zone, or recovery zone, of the device. For example, a first reservoir may be fluidically connected to the entry zone and a second reservoir may be fluidically connected to the recovery zone, e.g., as a source of recovery buffer.

In some embodiments of any of the preceding aspects, the cross-section of the active zone is selected from the group consisting of cylindrical, ellipsoidal, polygonal, star, parallelogram, trapezoidal, and irregular.

In some cases, the hydraulic diameter of the entry zone or the hydraulic diameter of the recovery zone is between 0.01% to 100,000% of the hydraulic diameter of the active zone. For example, the hydraulic diameter of the entry zone or the hydraulic diameter of the recovery zone may be 0.01% to 1000% of the hydraulic diameter of the active zone, e.g., 0.01% to 1%, 0.1% to 10%, 5% to 25%, 10% to 50%, 10% to 1000%, 25% to 75%, 25% to 750%, or 50% to 1000% of the hydraulic diameter of the active zone. Alternatively, the hydraulic diameter of the entry zone or the hydraulic diameter of the recovery zone may be 100% to 100,000% of the of the hydraulic diameter of the active zone, e.g., 100% to 1000%, 500% to 5,000%, 1,000% to 10,000%, 5,000% to 25,000%, 10,000% to 50,000%, 25,000% to 75,000%, or 50,000% to 100,000% of the hydraulic diameter of the active zone.

In some embodiments of any of the preceding aspects, the hydraulic diameter of the active zone is between 0.01 mm and 50 mm. In some embodiments, the length of the active zone is between 0.01 mm and 50 mm. In particular embodiments, the length of the active zone is between 0.01 mm and 25 mm. In some embodiments, the hydraulic diameter of any of the first electrode or the second electrode is between 0.1 mm to 500 mm. In particular embodiments, none of the entry zone, recovery zone, or active zone reduce a cross-section dimension of any of the plurality of cells suspended in the fluid, e.g., cells can pass through the device without deformation.

In further embodiments, the device includes an outer structure having a housing configured to encase the first electrode, second electrode, and the active zone of the device. In some embodiments, the outer structure is integral to the device. In certain embodiments, the outer structure is releasably connected to the device.

In some embodiments of any of the preceding aspects, the cross-section of the active zone is selected from the group consisting of cylindrical, ellipsoidal, polygonal, star, parallelogram, trapezoidal, and irregular.

In some embodiments of any of the preceding aspects, the hydraulic diameter of the entry zone or the hydraulic diameter of the recovery zone is between 0.01% to 100,000% of the hydraulic diameter of the active zone. For example, the hydraulic diameter of the entry zone or the hydraulic diameter of the recovery zone may be 0.01% to 1,000% of the hydraulic diameter of the active zone, e.g., 0.01% to 1%, 0.1% to 10%, 5% to 25%, 10% to 50%, 10% to 1,000%, 25% to 75%, 25% to 750%, or 50% to 100% of the hydraulic diameter of the active zone. Alternatively, the hydraulic diameter of the entry zone or the hydraulic diameter of the recovery zone may be 100% to 100,000% of the of the hydraulic diameter of the active zone, e.g., 100% to 1000%, 500% to 5,000%, 1,000% to 10,000%, 5,000% to 25,000%, 10,000% to 50,000%, 25,000% to 75,000%, or 50,000% to 100,000% of the hydraulic diameter of the active zone.

In some embodiments of any of the preceding aspects, the hydraulic diameter of the active zone is between 0.01 mm and 50 mm. In some embodiments, the length of the active zone is between 0.005 mm and 50 mm. In particular embodiments, the length of the active zone is between 0.005 mm and 25 mm. In some embodiments, the hydraulic diameter of any of the first electrode or the second electrode is between 0.1 mm to 500 mm. In particular embodiments, none of the entry zone, recovery zone, or active zone reduce a cross-section dimension of any of the plurality of cells suspended in the fluid, e.g., cells can pass through the device without deformation.

In some embodiments of any of the preceding aspects, the first and/or second electrodes is porous or a conductive fluid (e.g., liquid).

In further embodiments, the device includes an outer structure having a housing configured to encase the first electrode, second electrode, and the active zone of the device. In some embodiments, the outer structure is integral to the device. In certain embodiments, the outer structure is releasably connected to the device.

In some embodiments of any of the preceding aspects, the invention provides a system for introducing by electro-mechanical transfection a composition into a plurality of cells suspended in a flowing fluid, the system including any device described herein and a source of electrical potential, where the first and second electrodes of the device are releasably connected to the source of electrical potential. In the system, the plurality of cells suspended in the fluid are porated upon entering the active zone.

In further embodiments, the device includes an outer structure having a housing configured to encase the first electrode, second electrode, and the active zone of the device. In some embodiments, the outer structure includes a first electrical input operatively coupled to the first electrode and a second electrical input operatively coupled to the second electrode. In some embodiments, the releasable connection between the first or second electrical inputs and the source of electrical potential is selected from the group consisting of a clamp, a clip, a spring, a sheath, a wire brush, mechanical connection, inductive connection, or a combination thereof.

In some embodiments, the outer structure is integral to the device. In certain embodiments, the outer structure is releasably connected to the device.

In some embodiments, any of the devices, systems, or methods of any of the previous aspects induces reversible pore formation. In particular embodiments, the electro-mechanical transfection is substantially non-thermal reversible electro-mechanical transfection.

In some embodiments, the releasable connection between the device and the source of electrical potential is selected from the group consisting of a clamp, a clip, a spring, a sheath, a wire brush, mechanical connection, inductive connection, or a combination thereof. In particular embodiments, the releasable connection between the device and the source of electrical potential is a spring.

In some embodiments, the system further includes one or more reservoirs, e.g., a first reservoir and a second reservoir, fluidically connected to a zone, e.g., the entry zone or recovery zone, of a device. For example, a first reservoir may be fluidically connected to the entry zone and a second reservoir may be fluidically connected to the recovery zone.

In further embodiments, the system includes a fluid delivery source fluidically connected to the entry zone, wherein the fluid delivery source is configured to deliver the plurality of cells suspended in the fluid through the entry zone to the recovery zone. In some embodiments, the delivery rate from the fluid delivery source is between 0.001 mL/min to 1,000 mL/min, e.g., 25 mL/min. In certain embodiments, the residence time of any of the plurality of cells suspended in the fluid is between 0.5 ms to 50 ms. In some embodiments, the conductivity of the fluid is between 0.001 mS/cm to 500 mS/cm, e.g., 1-20 mS/cm.

In further embodiments, the system includes a controller operatively coupled to the source of electrical potential to deliver voltage pulses to the first electrode and second electrodes to generate an electrical potential difference between the first and second electrodes. In some embodiments, the voltage pulses have an amplitude of up to 3 kV, e.g., 0.01 kV to 3 kV, e.g., 0.2-0.6 kV. In some cases, the duty cycle of the electro-mechanical transfection is between 0.001% to 100%, e.g., 10-95%. In some embodiments, the voltage pulses have a duration of between 0.01 ms to 1,000 ms, e.g., 1-10 ms. In certain embodiments, the voltage pulses are applied the first and second electrodes at a frequency between 1 Hz to 50,000 Hz, e.g., 100-500 Hz. The waveform of the voltage pulse may be DC, square, pulse, bipolar, sine, ramp, asymmetric bipolar, arbitrary, or any superposition or combination thereof. In particular embodiments, the electric field generated from the voltage pulses has a magnitude of between 1 V/cm to 50,000 V/cm, e.g., 100-1,000 V/cm, e.g., between 400-1,000 V/cm. In further embodiments, the system includes a housing (e.g., a housing structure) configured to house the electro-mechanical transfection device described herein. In further instances, the housing (e.g., housing structure) includes a thermal controller configured to increase or decrease the temperature of the housing or any component of the system thereof. In some embodiments, the thermal controller is a heating element, e.g., a heating block, liquid flow, battery powered heater, or a thin-film heater. In other embodiments, the thermal controller is a cooling element, e.g., liquid flow, evaporative cooler, or a thermoelectric, e.g., a Peltier, device.

In further embodiments, the system includes a plurality of electro-mechanical transfection devices, e.g., in series or in parallel. In particular embodiments, the system includes a plurality of outer structures for the plurality of electro-mechanical transfection devices.

In further embodiments, the method includes assessing the health of a portion of the plurality of cells suspended in the fluid. In certain embodiments, the assessing includes measuring the viability of the portion of the plurality of cells suspended in the fluid. In some embodiments, the assessing includes measuring the transfection efficiency of the portion of the plurality of cells suspended in the fluid. In some embodiments, the assessing includes measuring the cell recovery rate of the portion of the plurality of cells suspended in the fluid. In certain embodiments, the assessing includes flow cytometry analysis of cell surface marker expression.

In some embodiments, the method induces reversible electro-mechanical transfection. In particular embodiments, the electro-mechanical transfection is substantially non-thermal reversible electro-mechanical transfection.

In some embodiments, cells suspended in the fluid with the composition are passed through the electric field in the active zone of the device by the application of a positive pressure, e.g., a pump, e.g., a syringe pump, peristaltic pump, or pressure source.

In certain embodiments, cells in the plurality of cells in the sample may be mammalian cells, eukaryotes, human cells, animal cells, plant cells, synthetic cells, primary cells, cell lines, suspension cells, adherent cells, unstimulated cells, stimulated cells, activated cells, immune cells, stem cells, blood cells, red blood cells, T cells, B cells, neutrophils, dendritic cells, antigen presenting cells (APCs), natural killer (NK) cells, monocytes, macrophages, or peripheral blood mononuclear cells (PBMCs), human embryonic kidney cells, e.g., HEK-293 cells, or Chinese hamster ovary (CHO) cells. In particular embodiments, the plurality of cells includes Jurkat cells. In particular embodiments, the plurality of cells includes primary human T-cells. In particular embodiments, the plurality of cells includes THP-1 cells. In particular embodiments, the plurality of cells includes primary human macrophages. In particular embodiments, the plurality of cells includes primary human monocytes. In particular embodiments, the plurality of cells includes natural killer (NK) cells. In particular embodiments, the plurality of cells includes Chinese hamster ovary cells. In particular embodiments, the plurality of cells includes human embryonic kidney cells. In particular embodiments, the plurality of cells includes B-cells. In particular embodiments, the plurality of cells includes primary human T-cells. In particular embodiments, the plurality of cells includes primary human monocytes. In particular embodiments, the plurality of cells includes primary human macrophages. In particular embodiments, the plurality of cells includes embryonic stem cells (ESCs), mesenchymal stem cells (MSCs), or hematopoietic stem cells (HSCs). In particular embodiments, the plurality of cells includes primary human induced pluripotent stem cells (iPSCs).

In some embodiments, the composition includes at least one compound selected from the group consisting of therapeutic agents, vitamins, nanoparticles, charged therapeutic agents, nanoparticles, charged molecules, e.g., ions in solution, uncharged molecules, nucleic acids, e.g., DNA or RNA, CRISPR-Cas complexes, proteins, polymers, ribonucleoproteins (RNPs), engineered nucleases, transcription activator-like effector nucleases (TALENs), zinc-finger nucleases (ZFNs), homing nucleases, meganucleases (MNs), megaTALs, enzymes, peptides, transposons, or polysaccharides, e.g., dextran, e.g., dextran sulfate. Compositions that can be delivered to cells in a suspension include nucleic acids (e.g., oligonucleotides, mRNA, or DNA), antibodies (or an antibody fragment, e.g., a bispecific fragment, a trispecific fragment, Fab, F(ab')2, or a single-chain variable fragment (scFv)), amino acids, polypeptides (e.g., peptides or proteins), cells, bacteria, gene therapeutics, genome engineering therapeutics, epigenome engineering therapeutics, carbohydrates, chemical drugs, contrast agents, magnetic particles, polymer beads, metal nanoparticles, metal microparticles, quantum dots, antioxidants, antibiotic agents, hormones, nucleoproteins, polysaccharides, glycoproteins, lipoproteins, steroids, analgesics, local anesthetics, anti-inflammatory agents, anti-microbial agents, chemotherapeutic agents, exosomes, outer membrane vesicles, vaccines, viruses, bacteriophages, adjuvants, vitamins, minerals, organelles, and combinations thereof. In certain embodiments, the composition is a nucleic acid (e.g., an oligonucleotide, mRNA, or DNA). In certain embodiments, the composition is an antibody. In certain embodiments, the composition is a polypeptide (e.g., a peptide or a protein).

In some embodiments, the composition has a concentration in the liquid of between 0.0001 μM and 20 μM (e.g., from 0.0001 μM to 0.001 μM, 0.001 μM to 0.01 μM, 0.001 μM to 5 μM, 0.005 μM to 0.1 μM, 0.01 μM to 0.1 μM, 0.01 μM to 1 μM, 0.1 μM to 1 μM, 0.1 μM to 5 μM, 1 μM to 10 μM, 1 μM to 15μM, or 1 μM to 20 μM, e.g., about 0.0001 μM, 0.0005 μM, 0.001 μM, 0.005 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 11 μM, 12 μM, 13 μM, 14 μM, 15 μM, 16 μM, 17μM, 18μM, 19 μM, or 20 μM).

In certain embodiments, the composition has a concentration in the fluid of between 0.0001 μg/mL and 1,000 μg/mL (e.g., from 0.0001 μg/mL to 0.001 μg/mL, 0.001 μg/mL to 0.01 μg/mL, 0.001 μg/mL to 5 μg/mL, 0.005 μg/mL to 0.1 μg/mL, 0.01 μg/mL to 0.1 μg/mL, 0.01 μg/mL to 1 μg/mL, 0.1 μg/mL to 1 μg/mL, 0.1 μg/mL to 5 μg/mL, 1 μg/mL to 10 μg/mL, 1 μg/mL to 50 μg/mL, 1 μg/mL to 100 μg/mL, 2.5 μg/mL to 15 μg/mL, 5 μg/mL to 25 μg/mL, 5 μg/mL to 50 μg/mL, 5 μg/mL to 500 μg/mL, 7.5 μg/mL to 75 μg/mL, 10 μg/mL to 100 μg/mL, 10 μg/mL to 1,000 μg/mL, 25 μg/mL to 50 μg/mL, 25 μg/mL to 250 μg/mL, 25 μg/mL to 500 μg/mL, 50 μg/mL to 100 μg/mL, 50 μg/mL to 250 μg/mL, 50 μg/mL to 750 μg/mL, 100 μg/mL to 300 μg/mL, 100 μg/mL to 1,000 μg/mL, 200 μg/mL to 400 μg/mL, 250 μg/mL to 500 μg/mL, 350 μg/mL to 500 μg/mL, 400 μg/mL to 1,000 μg/mL, 500 μg/mL to 750 μg/mL, 650 μg/mL to 1,000 μg/mL, or 800 μg/mL to 1,000 μg/mL, e.g., about 0.0001 μg/mL, 0.0005 μg/mL, 0.001 μg/mL, 0.005 μg/mL, 0.01 μg/mL, 0.02 μg/mL, 0.03 μg/mL, 0.04 μg/mL, 0.05 μg/mL, 0.06 μg/mL, 0.07 μg/mL, 0.08 μg/mL, 0.09 μg/mL, 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2 μg/mL, 2.5 μg/mL, 3 μg/mL, 3.5 μg/mL, 4 μg/mL, 4.5 μg/mL, 5 μg/mL, 5.5 μg/mL, 6 μg/mL, 6.5 μg/mL, 7 μg/mL, 7.5 μg/mL, 8 μg/mL, 8.5 μg/mL, 9 μg/mL, 9.5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, or 1,000 μg/mL).

In further embodiments, the method includes a housing structure configured to house the electro-mechanical device described herein. In further instances, the housing structure includes a thermal controller configured to increase or decrease the temperature of the housing or any component of the system thereof. In some embodiments, the thermal controller is a heating element, e.g., a heating block, liquid flow, battery powered heater, or a thin-film heater. In other embodiments, the thermal controller is a cooling element, e.g., liquid flow, evaporative cooler, or thermoelectric, e.g., Peltier device. In certain embodiments, the temperature of the plurality of cells suspended in the fluid is between 0° C. and 50° C.

In further embodiments, the device includes a plurality of electro-mechanical transfection devices, e.g., in series or in parallel. In particular embodiments, the device includes a plurality of outer structures for the plurality of devices.

In some embodiments, the method further includes storing the plurality of cells suspended in the fluid in a recovery buffer after transfection. In certain embodiments, the transfected cells have a viability after introduction of the composition between 0.1% and 99.9%, e.g., 75% and 95%. In other embodiments, the efficiency of the introduction of the composition into the cells is between 0.1 and 99.9%, e.g., between 25% and 95%. In certain embodiments, the cell recovery rate is between 0.1% and 100%. In particular embodiments, the cell recovery yield is between 0.1% and 500%.

In another aspect, the invention provides a kit for introducing by electro-mechanical transfection a composition into a plurality of cells suspended in a fluid, the kit including a plurality of devices as described herein, a plurality of outer structures as described herein, and a transfection buffer.

In another aspect, the invention provides a kit for electro-mechanical transfection of a composition into a plurality of cells suspended in a fluid, the kit including a plurality of devices as described herein, a plurality of outer structures as described herein, and a transfection buffer.

In some embodiments of any of the preceding aspects, the outer structures are integral to the plurality of cell devices. In certain embodiments, the outer structures are releasably connected to the plurality of devices.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “about,” as used herein, refers to ±10% of a recited value.

The term “average flow velocity,” as used herein refers to a velocity of a flowing liquid (e.g., in a channel or lumen) determined from the quotient of the volumetric flow rate (Q, units of m³/s) of the liquid (e.g., from a fluid delivery source, e.g., a pump) divided by the cross-sectional area (A, units of m²), e.g., of the channel or lumen in which the liquid flows, thus the average flow velocity (u) has units of m/s.

The term “plurality,” as used herein, refers to more than one.

The term “conductivity,” as used herein, refers to electrical conductivity, i.e., the ability of electrically charged particles (e.g., ions) to move through a medium, e.g., ions of a salt in the flowing liquid, e.g., buffer ions.

The term “substantially uniform,” as used herein, refers to +/−5% variance.

The term “minimum hydraulic diameter,” as used herein, refers to a length equal to the minimum quotient of four times the cross-sectional area divided by the wetted perimeter (e.g., the internal perimeter) of the cross-section e.g., of a lumen (e.g., a lumen of an active zone or entry zone).

The term “cross-sectional area,” unless otherwise specified, refers to the transverse cross-sectional area (e.g., along the plane perpendicular to the longitudinal axis or direction of flow).

The term “fluidically connected,” as used herein, refers to a direct connection between at least two device elements, e.g., an electro-mechanical device, a reservoir, etc., that allows for fluid to move between such device elements without passing through an intervening element.

The term “fluidic communication,” as used herein, refers to an indirect connection between at least two device elements, e.g., an active zone, a reservoir, etc., that allows for fluid to move between such device elements, e.g., through an intervening element, (e.g., through intervening tubing, an intervening channel, etc.).

The term “lumen,” as used herein, refers to an interior cavity of a portion of the devices of the invention (e.g., an active zone or entry zone) that allows for fluid to pass through.

The term “entry zone,” as used herein, comprises a portion of the devices of the invention through which a fluid and a plurality of cells suspended in the fluid may pass prior to electro-mechanical transfection in the active zone. An entry zone may further comprise an additional reservoir in fluidic communication with the active zone of the devices of the invention. When an electric potential difference is applied to a first and second electrode of the devices of the invention, the electric field that may be generated within an entry zone of the devices of the invention is not high enough to cause cell poration to occur.

The term “recovery zone,” as used herein, comprises a portion of the devices of the invention through which a fluid and a plurality of cells suspended in the fluid may pass or reside after electro-mechanical transfection in the active zone. A recovery zone may include a portion (e.g., a lumen, tube, channel, reservoir, etc.) of the device downstream of the active zone (e.g., immediately downstream, e.g., proximal to the second outlet). A recovery zone may further comprise an additional reservoir in fluidic communication with the active zone.

The term “active zone,” as used herein, refers to a portion of a device that is disposed between first and second electrodes, and in fluidic communication with, and downstream of, the entry zone (e.g., downstream of a first outlet). The electric field is delivered to the fluid in the active zone.

The term “transfection,” as used herein, refers to a process by which payloads can be introduced into cells utilizing means other than viral delivery methods, such as biological, chemical, electrical, mechanical, or physical methods.

The term “electroporation,” as used herein, refers to a process utilizing applied electric fields to create small pores in cell membranes through which payloads can be introduced into cells (e.g., as a method of transfection).

The term “electro-mechanical transfection,” as used herein, refers to a transfection process by which payloads can be introduced to cells utilizing a combination of an applied electric field and a mechanical poration mechanism. This delivery method has the potential to decrease and/or stabilize the overall electric field exposure of the cells in the active zone, thereby enhancing cell viability and/or transfection efficiency, or both. The devices of the invention are configured to transfect cells via electro-mechanical transfection rather than by electroporation alone. Methods of the invention allow the optimum combination of electrical energy (e.g., electric field strength) and mechanical energy (e.g., flow rate) to be determined for a given cell type.

The term “therapeutic dose,” as used herein, refers to a quantity of transfected cells that, when administered to a patient for treating a state, disorder, or condition, is sufficient to achieve such treatment. The therapeutic dose administration may be alone, in combination with other agents, as part of a series of administrations, or a combination thereof. The treatment may produce a therapeutic benefit, e.g., a beneficial immune response, a reduction or elimination of a disease state, diminution of, e.g., cancer cells or cancer biomarkers, a slowing or arresting cancer cell replication rate, etc. A therapeutic dose may be determined by, e.g., monitoring of patient condition (e.g., by clinical assessment), clinical disease progression, quantities of disease or organ function biomarkers, quantities of, e.g., white or red blood cells, etc. A therapeutic dose may be any quantity or concentration of cells described herein.

### BRIEF DESCRIPTION OF THE DRAWINGS

FIG. 1 shows a schematic of a device of the invention. Cells and payload are suspended in proprietary buffer in the reservoir. As the cells and payload flow through the electro-mechanical transfection zone they are exposed to both electrical energy and continuous fluid flow to induce transient cell membrane disruption and simultaneous delivery of genetic payloads into the cells. Transfected cells are then dispensed directly into growth media for cell recovery.

FIGS. 2A-2D show parameters associated with electro-mechanical transfection correlated with outcome data. Expanded human T cells, transfected with a GFP reporter mRNA using devices, systems, and methods of the invention. Cultures were assessed for cell viability (7AAD negative), transfection efficiency, and percent yield (observed live GFP⁺ cells from 1 e6 input cells) at 24 hours. The mechanism of action for transfection in GFP reporter mRNA delivery to expanded T cells according to methods of the invention is visualized here by plotting percent viability, efficiency, and yield against the electro-mechanical transfection specific parameter π₄ (FIGS. 2A and 2B) and π₅ (FIGS. 2C and 2D). All analysis was completed using the Thermo Fisher Attune™ NxT flow cytometer; n=89 depicted as individual data points.

FIGS. 3A-3F show comparison data of transfection using an electro-mechanical device according to methods of the invention vs two commercial electroporation-based transfection systems (Neon™ and 4D Nucleofector™). Expanded human T cells were processed without payload using an electro-mechanical transfection system or commercially available electroporation-based transfection systems (Neon™ and 4D Nucleofector™). Representative data is shown here at 6 or 24 hours post processing. In FIGS. 3A-3C, volcano plots showing significantly dysregulated genes (p<0.05) with greater than 1-fold change in expression at 6 hours. FIG. 3D is a graphical representation of genes exhibiting baseline versus dysregulated expression at 6 hours. FIG. 3E is a heatmap of selected up and down regulated genes at 6 and 24 hours. FIG. 3F is a heatmap of gene ontology focused on T cell function.

FIGS. 4A-4B show results of using an electro-mechanical transfection system according to methods of the invention across different donors vs a control. Expanded human T cells from three unique donors were transfected with a GFP reporter mRNA. Cultures were assessed for cell viability (7AAD negative) (FIG. 4A) and transfection efficiency (FIG. 4B) at 24 hours. Bar graphs are Mean±SD with the following transfection sample sizes Donor #1 n=3, Donor #2 n=22, Donor #3 n=3.

FIGS. 5A-5F show the results of using an electro-mechanical transfection system and the method of the invention to transfect naive T cells. Naïve T cells were transfected with a GFP reporter mRNA. Cultures were assessed for expansion capability (FIG. 5A) and cell viability (FIG. 5B) (Trypan blue exclusion) measured out to 6 days after transfection. FIG. 5C shows a comparison of naïve T cells stained for expression of lineage markers CD45RA and CD45RO vs a control. FIGS. 5D-5F show cell viability (7AAD negative) (FIG. 5D), transfection efficiency (FIG. 5E), and live GFP+ cell count (yield) (FIG. 5F) from 0.5 M cell input measured at 24 hours after transfection. Bar graphs are representative of n=2 transfections Mean±SD.

FIGS. 6A-6B demonstrate how methods of the invention and electro-mechanical transfection as described herein can directly translate from small-scale research transfections to large-scale cell manufacturing transfections. FIG. 6A is a schematic showing how the electro-mechanical flow cell can be used in a small-scale device (e.g., integrated with a liquid handling machine into a 96-well format) electro-mechanical transfection (e.g., an array of devices disposed for batchwise transfection) and a large-scale electro-mechanical device or system (e.g., a closed electro-mechanical transfection system), allowing for direct translation from one scale to the other. Expanded human T cells were transfected with GFP mRNA reporter payload using a small volume electro-mechanical device array platform (small scale 10-100 μL transfections) and a large volume flow electro-mechanical transfection system (large scale >5 mL transfections). The cells were assessed for cell viability (7AAD negative) (FIG. 6B) and transfection efficiency (FIG. 6B) at 24 hours. Bar graphs are Mean±SD small volume n=6 large volume n=2.

FIGS. 7A-7B show a gating strategy for determining total cell counts and viability. In FIG. 7A total cells are pre-gated in the forward scatter (FSC) and side scatter (SSC) dot pots. This gate captures cells of broad morphologies for accurate analysis of the total cell population. In FIG. 7B, the viability is then determined by gating 7-AAD- cells from within the total cell gate. Efficiency gates are determined based on nontreated cells to eliminate any background fluorescence (not shown).

FIGS. 8A-8B are heatmaps of field strength vs flow rate with viability (FIG. 8A) and efficiency (FIG. 8B) in the z-axis. Expanded human T cells were transfected with a GFP reporter mRNA using a large volume transfection system. Cultures were assessed for cell viability (FIG. 8A) (7AAD negative) and transfection efficiency (FIG. 8B) at 24 hours. All analysis was completed using the Thermo Fisher Attune™ NxT flow cytometer; n=96 depicted as individual data points

FIGS. 9A-9C are volcano plots for expanded human T cells from the second of two donor unique donors processed without payload using electro-mechanical (FIG. 9A) or commercially available electroporation-based transfection systems: Neon™ (FIG. 9B) and 4D Nucleofector™ (FIG. 9C). The volcano plots show significantly dysregulated genes (p<0.05) with greater than 1-fold change in expression at 6 hours post processing.

FIGS. 10A-10B are bar graphs showing cell viability (7AAD negative) (FIG. 10A) and transfection efficiency (FIG. 10B) at 24 hours for expanded human T cells transfected with GFP reporter mRNA using an electro-mechanical transfection system or commercially available electroporation-based transfection systems (Neon™ and 4D Nucleofector™). Bar graphs are Mean±SD n=7.

FIGS. 11A-11C show use of a system of the invention to deliver multiple payloads. Expanded human T cells were transfected with GFP reporter mRNA and mCherry reporter mRNA a system including an array of devices of the invention. Cultures were assessed for cell viability (7AAD negative) (FIG. 11A) and transfection efficiency (FIGS. 11B and 11C) at 24 hours after either in parallel (FIG. 11B) or in series (separated by 48 hours) (FIG. 11C) delivery.

### DETAILED DESCRIPTION OF THE INVENTION

The present invention provides methods for the transfection of cells, e.g., mammalian cells, e.g., primary T cells, by electro-mechanical transfection at equal or greater volumes, equal or higher transfection efficiencies, equal or higher throughputs, equal or higher recovery rates, equal or higher yields, and equal or higher cell viabilities as compared with traditional cuvette-based electroporation approaches or commercially available electroporation instruments. In particular, systems and methods are provided that can perform electro-mechanical transfection in a flow-through manner, a continuous manner, or using a plurality of electro-mechanical transfection devices of the invention to enhance throughput and cell numbers. More particularly, the methods of the invention allow less electrical energy to be applied than in electroporation-based techniques, thus minimizing damage to transfected cells.

Non-viral cell transfection represents a promising evolution of both autologous and allogenic cell therapy. Despite the advantages of separating cell therapy from viral vector production challenges, non-viral transfection has lagged behind viral methodologies in clinical applications. One of the most well-known forms of non-viral transfection is electroporation, where a high energy electric field is applied to a static cell suspension. Successful transfection via classical electroporation is dependent on the strength of the electric field each cell experiences. However, electric fields that are too intense over an extended period of time can result in irreversible cell membrane disruption, leading to cell death.

To improve the yield of electric field assisted transfection, the electrical energy applied to the cells must be minimized. Methods of the invention reduce the electrical energy required to enable cell membrane permeability by adding a mechanical component to the total energy applied to the cells. This mechanical energy can be delivered via, e.g., fluid flow, reducing the high energy electric fields needed for efficient delivery of genetic payloads by inducing membrane disruption for delivery of, e.g., DNA, RNA, or CRISPR-RNP into the nucleus.

Electro-mechanical cell transfection involves the use of electric fields coupled with mechanical stress, e.g., associated with moderate fluid flow rates, to permeate cells and deliver exogenous material. This technique is distinct from electroporation, where an electric field alone is utilized to permeate cells, typically with no flow or at low fluid flow rates that result in minimal stress. In the case of electro-mechanical cell transfection, pore formation is mediated by the combined effects of the electric field and the mechanical energy input in the form of shear and normal stresses on the cell. One would expect that electro-mechanical cell transfection would depend upon the following parameters; the root-mean-square of the applied voltage, V_{RMS}; medium conductivity (i.e., electrical conductivity), s; average fluid velocity, u; the distance between electrodes, l; dynamic viscosity of the fluid (e.g., as measured by a rotational viscometry), μ; the channel diameter, d; the cell diameter, D; and the fluid density, r. Applying the Buckingham Pi theorem for dimensional analysis, we obtain a set of four dimensionless parameters. The first two parameters

$${Π}_{1} = \frac{l}{d} and {Π}_{2} = \frac{D}{d} ,$$

are dimensionless lengths that are scaled by the cell diameter. The third dimensionless group is the classic Reynolds Number,

$$Re = \frac{ρ ud}{μ} ,$$

which is the ratio of inertial effects to viscous effects in the fluid flow. Electro-mechanical transfection occurs at moderate Re on the order 10² and thus falls in the laminar flow regime. The fourth dimensionless group,

$${Π}_{4} = \frac{σ {V}_{R M S}^{2}}{μ {u}^{2}} ,$$

represents the ratio of electrical power applied to mechanical power imposed on the cell suspension. Dynamic viscosity (p) may be determined by, e.g., a rotational viscometer, e.g., as described in Pries et al., “Blood viscosity in tube flow: dependence on diameter and hematocrit.” American Journal of Physiology-Heart and Circulatory Physiology 263.6 (1992): H1770-H1778.

We expect that the key physics of this process will be governed by these four dimensionless groups and combinations thereof. The existence and importance of Re and π₄ distinguish this transfection mechanism from both electroporation and purely mechanical based transfection methods. In electroporation, the electric field and pulse conditions govern the transfection efficiency and the process typically occurs in static chambers with a quiescent fluid. There are recent efforts involving flow-based electroporation in which the cell suspension moves with a finite velocity during the transfection process. However, in these systems the flow is used to deliver cells to the transfection zone, not to influence the transfection itself, as is the case with electro-mechanical transfection. Mechanical based transfection methods, particularly those that employ higher flow rates would surely be dependent upon Re but as there is no applied electric field there is no need for the π₄ described here. Therefore, despite some superficial similarities to electroporation, electro-mechanical transfection is a unique and novel technique to deliver exogenous material to cells.

The invention presents a new transfection technology, electro-mechanical transfection utilizing electrical energy with continuous flow that demonstrates several advantages over electroporation and other viral and non-viral transfection methodologies. Dimensional analysis reveals that electro-mechanical transfection is optimized by balancing the effects of fluid flow and electric fields, distinguishing this technology from previous methods employing electric fields. The physical model presented illuminates the critical parameters driving the effect of this technology, mainly defined by combining the previous dimensionless parameter into a fifth dimensionless group,

$${Π}_{5} = \frac{( R e ) ( {Π}_{4} )}{{Π}_{1}^{2}} = \frac{ρ σ {d}^{3}}{{μ}^{2}} ( \frac{{E}^{2}}{u} ) ,$$

containing the ratio of the electric field squared and the channel velocity. Optimizing payload delivery efficiency to cells of interest while maintaining viability (e.g., to optimize engineered cell yield), is the goal of a transfection solution and the invention provides effective methods for rapid optimization of the key parameters for effective electro-mechanical transfection.

The transcriptome analysis results described herein show that high delivery efficiency can be decoupled from significant gene dysregulation. Electro-mechanical transfection according to the invention exhibited less than a 5% shift from baseline 6 hours after processing while both commercially available electroporation-based transfection devices exhibited greater than 5% shift from baseline in total gene dysregulation. Of the dysregulation induced by non-viral processing only 13% could be attributed to altered molecular function in the electro-mechanical device while electroporation-based devices induced between 19-24% dysregulation attributed to molecular function. This functional dysregulation was highlighted when markers of T cell exhaustion (CTLA4 and TIG IT) were found to be upregulated 6 hours after processing with electroporation-based devices but found to be at baseline levels after processing with the electro-mechanical device according to methods of the invention. Analysis at 24 hours for transfection efficiency showed that the reduced gene dysregulation observed after electro-mechanical processing resulted in delivery efficiency metrics that were not significantly different form the commercially available electroporation-based device from Thermo Fisher (Neon™), e.g., 89.2% and 89.4%, respectively. The invention affords post transfection viability of greater than 75% and delivery efficiency greater than 80% was observed in multiple use cases. These findings were also confirmed in multiple PBMC donors with no significant difference in delivery efficiency, with a range of 84.0% to 93.7% in efficiency (GFP⁺ live cells) observed between all three donors. Additionally, the observed high transfection efficiencies did not result in altered cell state as indicated in this study by maintenance of lineage specific naïve cell marker expression (CD45RA⁺/CD45RO⁻). These findings showed that high viability and delivery efficiency, both above 95%, could be maintained in naïve CD4+ T cells 24-hours after electro-mechanical transfection while maintaining retention of naïve marker expression, 100% CD45RA⁺/CD45RO⁻. Moreover, results from the 50-fold scale-up transfection resulted in ≤2.5% change in both viability and delivery efficiency compared to the small-scale results. Together this data suggests that cell engineering using non-viral electro-mechanical transfection methods are distinct from classical electroporation and represent a meaningful alternative to existing transfection methods. Electro-mechanical transfection can be leveraged with high throughput automation for discovery or process development, while also easily scaling up for manufacturing. This ability to scale out and scale up, while maintaining cell health and high cell yield, make electro-mechanical transfection an attractive new solution for cell therapy development and manufacturing.

### Devices

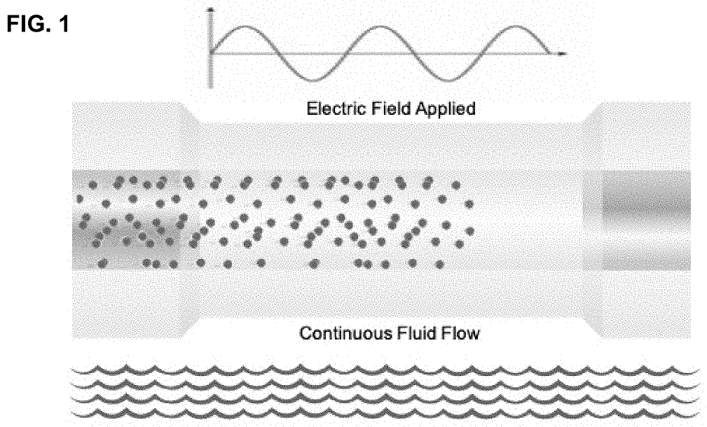

In general, devices of the present invention are configured to be flow through devices that may interface with existing liquid handling, pumps, or fluid transport apparatuses, such as conventional pipette tip robots or large-scale liquid handling systems, to provide continuous transfection of cells suspended in a fluid. A device of the invention is configured for transfection of cells to occur within the active zone via an electro-mechanical transfection mechanism that is distinct from the delivery mechanism in electroporation-based transfection systems. Devices of the invention typically feature two distinct regions: an entry zone, with a first inlet and first outlet, and an active zone with a second inlet and second outlet. First and second electrodes are disposed to produce an electric field in the active zone. An example of an embodiment of the device of the invention is shown in FIG. 1. When an electrical potential difference is applied to the first and second electrodes, a localized electric field develops in the space between the two electrodes, e.g., the active zone, and cells that are exposed to the electric field are porated. An individual device of the invention may include two electrodes, as shown in FIG. 1; alternatively, individual devices of the invention may include three or more electrodes that define a plurality of active zones, thus allowing for a plurality of transfections on the cells suspended in a fluid. Devices of the invention may include a plurality of active zones between the first and second electrodes, allowing for cells to experience different electric fields, e.g., developed by different geometries of each of the plurality of active zones, while flowing in a single device or a plurality of devices.

In some cases, the first electrode and the second electrode may be electrically conductive wires, hollow cylinders, electrically conductive thin films, metal foams, mesh electrodes, liquid diffusible membranes, conductive liquids, or any combination thereof can be included in the device. The electrodes may be either aligned parallel with the axis of fluid flow of the device or may be aligned orthogonal to the axis of fluid flow of the device. For example, the first and second electrodes may be hollow cylindrical electrodes arranged in parallel with the axis of fluid flow within the device, such as the in the device of FIG. 1, such that fluid flows through the electrodes. In an alternative example, the first and/or second electrodes may be made of a porous conductor, e.g., a metal mesh, with pores that are aligned to the axis of fluid flow of the device. In an alternative example, the first and/or second electrodes may be a conductive fluid, e.g., liquid. In some cases, the first and second electrodes may be configured as a helical, e.g., a double helix, made of a solid conductor, e.g., a wire, around the active zone. In this configuration, the hydraulic diameter of the active zone remains substantially uniform but the first and second electrodes change in position along the length of the active zone. The first and second electrodes are in fluid communication with the active zone but the electric field generated when an electrical potential difference is applied to the electrodes rotates as the cells suspended in the fluid travel through the device of the invention. In certain embodiments, the first and second electrodes are embedded into the device of the invention and have active area disposed at or near the fluidic connections to the active zone such that the fluid carrying the cells in suspension contacts a portion of the electrode, with the electric field generated in the active zone.

When configured to be hollow cylindrical electrodes, the diameter of the electrode may be from about 0.1 mm to 5 mm, e.g., from about 0.1 mm to 1 mm, from 0.5 mm to 1.5 mm, from 1 mm to 2 mm, from 1.5 mm to 2.5 mm, 2 mm to 3 mm, from 2.5 mm to 3.5 mm, 3 mm to 4 mm, from 3.5 mm to 4.5 mm, or 4 mm to 5 mm, e.g., 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, or 5 mm. An exemplary electrode outer diameter is 1.3 mm, corresponding to a 16 gauge electrode.

The active zone may fluidically and/or electrically connect the first and second electrodes of devices of the invention, and when the electrodes are energized, experiences a localized electric field therebetween. The active zone may be fluidically connected to a recovery zone downstream of the active zone. The cross-sectional shape of the active zone may be of any suitable shape that allows cells to pass through the active zone and the electric field within the active zone. The cross-sectional shape may be, e.g., circular, ellipsoidal, or polygonal, e.g., square, rectangular, triangular, n-gon (e.g., a regular or irregular polygon having 4, 5, 6, 7, 8, 9, 10, or more sides), star, parallelogram, trapezoidal, or irregular, e.g., oval, or curvilinear shape. In some cases, the active zone is a channel that has a substantially uniform cross-section dimension along its length, e.g., the active zone may have a circular cross-section, where the diameter is constant from the fluidic connection with the entry zone to the fluidic connection of the outlet (e.g., the second outlet) of the active zone, or of the recovery zone. In this configuration, the resulting electric field is more uniform, thus allowing for a more predictable electric field exposure of cells suspended in a fluid. Alternatively, the hydraulic diameter of the active zone may be varied along is length. For example, the hydraulic diameter of the active zone may either increase or decrease along its length, or may have more than one dimension change along its length, e.g., the hydraulic diameter, e.g., the diameter, may increase or decrease by at least about 1%, 5%, 10%, 20%, 30%, 40%, 50%, 60%, 70%, 80%, 90%, or 100%, or at most about 1%, 5%, 10%, 20%, 30%, 40%, 50%, 60%, 70%, 80%, 90%, or 100%. In this configuration, the active zone may have a truncated conical cross-section, with the diameter increasing from the top aperture to the bottom aperture or decreasing from the top aperture to the bottom aperture. In some cases, devices of the invention may include a plurality of active zones fluidically connected in series, with each active zone having either a uniform or non-uniform cross-section and each may have a different cross-section shape. As a non-limiting example, a device of the invention may include a plurality of serially-connected active zones, each of the plurality of active zones having a cylindrical cross-section of a different hydraulic diameter, e.g., each has a different diameter.

In some embodiments, the hydraulic diameter of the active zone may be from 0.005 mm to 50 mm, e.g., 0.005 mm to 0.05 mm, 0.01 mm to 0.1 mm, 0.05 mm to 0.5 mm, 0.1 mm to 1 mm, 0.5 mm to 1 mm, from 0.5 mm to 2 mm, 0.7 mm to 1.5 mm, 1 mm to 5 mm, 3 mm to 7 mm, 5 mm to 10 mm, 7 mm to 12 mm, 10 mm to 15 mm, 13 mm to 18 mm, 15 mm to 20 mm, 22 mm to 30 mm 25 mm to 35 mm, 30 mm to 40 mm, 35 mm to 45 mm, or 40 mm to 50 mm, e.g., about 0.005 mm, 0.006, 0.007 mm, 0.008 mm, 0.009 mm, 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm. In general, the diameter of the active zone is sized such that it does not have a constriction that contacts the cells to deform the cell membranes with the channel walls, e.g., poration of the cells is not induced by mechanical deformation due to cell squeezing, e.g., the cells can freely pass through the active zone.

In some cases, the length of the active zone may be from 0.005 mm to 50 mm, e.g., 0.005 mm to 0.05 mm, 0.01 mm to 0.1 mm, 0.05 mm to 0.5 mm, 0.1 mm to 1 mm, from 0.5 mm to 2 mm, 1 mm to 5 mm, 3 mm to 7 mm, 4 mm to 8 mm, 5 mm to 10 mm, 7 mm to 12 mm, 10 mm to 15 mm, 13 mm to 18 mm, 15 mm to 20 mm, 22 mm to 30 mm 25 mm to 35 mm, 30 mm to 40 mm, 35 mm to 45 mm, or 40 mm to 50 mm, e.g., about 0.005 mm, 0.006, 0.007 mm, 0.008 mm, 0.009 mm, 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm.

The hydraulic diameter of the entry zone and/or the recovery zone may be independently substantially the same as the hydraulic diameter of the active zone. Alternatively, the entry zone and/or the recovery zone may be independently smaller or larger than the hydraulic diameter of the active zone. For example, when the hydraulic diameter of the entry zone and/or the recovery zone is independently configured to be smaller than the hydraulic diameter of the active zone, the hydraulic diameter of the entry zone and/or the recovery zone may be from 0.01% to 100% of the hydraulic diameter of the active zone, 0.01% to 1%, 0.1% to 10%, 1% to 5%, 1% to 10%, 5% to 25%, 5% to 10%, 10% to 25%, 10% to 50%, 25% to 75%, or 50% to 100%, e.g., about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%.

Alternatively, when the hydraulic diameter of the entry zone and/or the recovery zone is independently configured to be larger than the hydraulic diameter of the active zone, the hydraulic diameter of the entry zone and/or the recovery zone may be from 100% to 100,000% of the hydraulic diameter of the active zone, e.g., 100% to 1000%, 100% to 250%, 100% to 500%, 250% to 750%, 500% to 1,000%, 500% to 5,000%, 1,000% to 10,000%, 5,000% to 25,000%, 10,000% to 50,000%, 25,000% to 75,000%, or 50,000% to 100,000%, e.g., about 100%, 150%, 175%, 200%, 225%, 250%, 300%, 250%, 400%, 450%, 500%, 600%, 700%, 800%, 900%, 1,000%, 2,000%, 3,000%, 4,000%, 5,000%, 6,000%, 7,000%, 8,000%, 9,000%, 10,000%, 15,000%, 20,000%, 25,000%, 30,000%, 35,000%, 40,000%, 45,000%, 50,000%, 55,000%, 60,000%, 65,000%, 70,000%, 75,000%, 80,000%, 85,000%, 90,000%, 95,000%, or 100,000%.

Devices of the invention may also include one or more reservoirs for fluid reagents, e.g., a buffer solution, or samples, e.g., a suspension of cells and a composition to be introduced to the cells. For example, devices of the invention may include a reservoir for the cells suspended in the fluid to flow into the entry zone and active zone and/or a reservoir for holding the cells that have been transfected. Similarly, a reservoir for liquids to flow in additional components of a device, such as additional inlets that intersect the first or second electrodes, may be present. A single reservoir may also be connected to multiple devices of the invention, e.g., when the same liquid is to be introduced at two or more individual device of the invention configured to transfect cells in parallel or in series. Alternatively, devices of the invention may be configured to mate with sources of the liquids, which may be external reservoirs such as vials, tubes, or pouches. Similarly, the device may be configured to mate with a separate component that houses the reservoirs. Reservoirs may be of any appropriate size, e.g., to hold 10 mL to 5000 mL, e.g., 10 mL to 3000 mL, 25 mL to 100 mL, 100 mL to 1000 mL, 40 mL to 300 mL, 1 mL to 100 mL, 10 mL to 500 mL, 250 mL to 750 mL, 250 mL to 1000 mL, or 1000 mL to 5000 mL. When multiple reservoirs are present, each reservoir may have the same or a different size.

In addition to the components discussed above, devices of the invention may include additional components. For example, the first and second electrodes of the devices of the invention may include one or more additional fluid inlets to allow for the introduction of non-sample fluids, e.g., buffer solutions, into the appropriate region of the device. For example, a recovery zone of a device of the invention may include an additional inlet and outlet to circulate a recovery buffer to aid in providing the cells a growth environment after the transfection process.

### Systems and Kits

One or more electro-mechanical transfection devices of the invention may be combined with various external components, e.g., power supplies, pumps, reservoirs (e.g., bags), controllers, reagents, liquids, and/or samples in the form of a system. In some embodiments, a system of the invention includes a plurality of devices of the invention and a source of electrical potential that is releasably connected to the first and second electrodes of the device(s) of the invention. In this configuration, the device(s) of the invention are connected to the source of electrical potential, and the first electrode is energized and the second electrode is held at ground. This creates a localized electric field in the active zone, thus transfecting the cells that pass through the device(s). Electro-mechanical systems incorporating devices of the invention may induce reversible poration of the cells that pass through the device and system of the invention. For example, devices and systems of the invention may induce substantially non-thermal reversible poration.

In some cases, the releasable connection to the first and second electrodes may include any practical electro-mechanical connection that can maintain consistent electrical contact between the source of electrical potential and the first and second electrodes. Example electrical connections include, but are not limited to clamps, clips, e.g., alligator clips, springs, e.g., a leaf spring, an external sheath or sleeve, wire brushes, flexible conductors, pogo pins, mechanical connections, inductive connections, or a combination thereof. Other types of electrical connections are known in the art. A device of the invention can be installed into an opening in the conducting grid such that the first and second electrodes of the device can contact the conducting grid. In particular, the conducting grid includes spring loaded electrodes, e.g., electrodes connected to a spring, such that when a device of the invention is installed into an opening of the conducting grid, the spring-loaded electrodes displace and compress the spring (which further provides a restoring force against the first and second electrodes of the device of the invention), thus ensuring electrical contact between the device of the invention and the source of electrical potential.

The source of electrical potential is configured to deliver an applied voltage to one or more electrode in order to provide an electrical potential difference between the electrodes and thus establish a uniform electric field in the active zone. In some cases, such as in a two-electrode circuit, the applied voltage is delivered to a first electrode and the second electrode is held at ground. Without wishing to be bound by any particular theory, an applied voltage delivered to the electrode is delivered at a particular amplitude, a particular frequency, a particular pulse shape, a particular duration, a particular number of pulses applied, and a particular duty cycle. These parameters, coupled to the geometry of the active zone, will deliver a particular electric field within the active zone that will be experienced by the cells suspended in a fluid. The electrical parameters described herein may be optimized for a particular cell line and/or composition being delivered to a particular cell line. The application of the electrical potential to the electrodes of devices(s) of the invention may be initiated and/or controlled by a controller, e.g., a computer with programming, operatively coupled to the source of electrical potential.

Along with the electrical potential parameters described herein, the geometry of devices of the invention, e.g., the shape and dimensions of the cross-section of the active zone, control the shape and intensity of the resulting electric field within the active zone. Typically, a device with an active zone that has a uniform cross section will exhibit a uniform electric field along its length. In order to modulate the resulting electric field in the active zone, the active zone may include a plurality of different hydraulic diameters and/or different cross-section shapes along its length. As a non-limiting example, a device of the invention may include a plurality of serially-connected active zones, each of the plurality of active zones having a circular cross-section of a different hydraulic diameter, e.g., each has a different diameter. In this configuration, the different diameter circular cross-sections of the active zone each act as an independent active zone, and each will induce a different electric field at every change in dimension with an identical applied voltage, e.g., a constant DC voltage.

In some cases, devices of the invention may include a plurality of active zones fluidically connected in series, with each active zone having either a uniform or non-uniform cross-section and each may have a different cross-section shape. Alternatively, a system of the invention may include a plurality of devices of the invention in a parallel configuration, with each device operating independently of each other to increase the overall throughput of the electro-mechanical transfection.

In some cases, the amplitude of the applied voltage is from −3 kV to 3 kV, e.g., −3 kV to −0.1 kV, −2 kV to −0.1 kV, −1 kV to −0.1 kV, −0.1 kV to −0.01 kV, 0.01 kV to 3 kV, e.g., 0.01 kV to 0.1 kV, 0.02 kV to 0.2 kV, 0.03 kV to 0.3 kV, 0.04 kV to 0.4 kV, 0.05 kV to 0.5 kV, 0.06 kV to 0.6 kV, 0.07 kV to 0.7 kV, 0.08 kV to 0.8 kV, 0.09 kV to 0.9 kV, 0.1 kV to 1 kV, 0.1 kV to 2.0 kV, 0.1 kV to 3 kV, 0.15 kV to 1.5 kV, 0.2 kV to 2 kV, 0.25 kV to 2.5 kV, or 0.3 kV to 3 kV, e.g., 0.01 to 1 kV, 0.1 kV to 0.7 kV, or 0.2 to 0.6 kV, e.g., about 0.01 kV, 0.02 kV, 0.03 kV, 0.04 kV, 0.05 kV, 0.06 kV, 0.07 kV, 0.08 kV, 0.09 kV, 0.1 kV, 0.2 kV, 0.3 kV, 0.4 kV, 0.5 kV, 0.6 kV, 0.7 kV, 0.8 kV, 0.9 kV, 1 kV, 1.1 kV, 1.2 kV, 1.3 kV, 1.4 kV, 1.5 kV, 1.6 kV, 1.7 kV, 1.8 kV, 1.9 kV, 2 kV, 2.1 kV, 2.2 kV, 2.3 kV, 2.4 kV, 2.5 kV, 2.6 kV, 2.7 kV, 2.8 kV, 2.9 kV, or 3 kV.

In some cases, the frequency of the applied voltage is from 1 Hz to 50,000 Hz, e.g., from 1 Hz to 1,000 Hz, 1 Hz to 500 Hz, 100 Hz to 500 Hz, 100 Hz to 5,000 Hz, 500 Hz to 10,000 Hz, 1000 Hz to 25,000 Hz, or from 5,000 Hz to 50,000 Hz, e.g., from 10 Hz to 1000 Hz, 10 Hz to 500 Hz, 500 Hz to 750 Hz, or 100 Hz to 500 Hz, e.g., from about 1 Hz, 2 Hz, 3 Hz, 4 Hz, 5 Hz, 6 Hz, 7 Hz, 8 Hz, 9 Hz, 10 Hz, 20 Hz, 30 Hz, 40 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, 200 Hz, 210 Hz, 220 Hz, 230 Hz, 240 Hz, 250 Hz, 260 Hz, 270 Hz, 270 Hz, 280 Hz, 290 Hz 300 Hz, 310 Hz, 320 Hz, 330 Hz, 340 Hz, 350 Hz, 360 Hz, 370 Hz, 380 Hz, 390 Hz, 400 Hz, 410 Hz, 420 Hz, 430 Hz, 440 Hz, 450 Hz, 460 Hz, 470 Hz, 480 Hz, 490 Hz, 500 Hz, 510 Hz, 520 Hz, 530 Hz, 540 Hz, 550 Hz, 600 Hz, 700 Hz, 800 Hz, 900 Hz, 1,000 Hz, 2,000 Hz, 3,000 Hz, 4,000 Hz, 5,000 Hz, 6,000 Hz, 7,000 Hz, 8,000 Hz, 9,000 Hz, 10,000 Hz, 15,000 Hz, 20,000 Hz, 25,000 Hz, 30,000 Hz, 35,000 Hz, 40,000 Hz, 45,000 Hz, or 50,000 Hz.

In some embodiments, the shape of the applied pulse, e.g., waveform, can be a square wave, pulse, a bipolar wave, a sine wave, a ramp, an asymmetric bipolar wave, or arbitrary. Other voltage waveforms are known in the art. The chosen waveform can be applied at any practical voltage pattern including, but not limited to, high voltage-low voltage, low voltage-high voltage, direct current (DC), alternating current (AC), unipolar, positive (+) polarity only, negative (−) polarity only, (+)/(−) polarity, (−)/(+) polarity, or any superposition or combination thereof. A skilled artisan can appreciate that these pulse parameters will depend on the cell line any electrical characteristics of the composition being delivered to the cell.

Applied voltage pulses can be delivered to the active zone with durations from 0.01 ms to 1,000 ms, e.g., from 0.01 ms to 1 ms, 0.1 ms to 10 ms, 0.1 ms to 15 ms, 1 ms to 10 ms, 1 ms to 50 ms, 10 ms to 100 ms, 25 ms to 200 ms, 50 ms to 400 ms, 100 ms to 600 ms, 300 ms to 800 ms, or 500 ms to 1,000 ms, e.g., about 0.01 ms to 100 ms, 0.1 ms to 50 ms, or 1 ms to 10 ms, e.g., 0.01 ms, 0.02 ms, 0.03 ms, 0.04 ms, 0.05 ms, 0.06 ms, 0.07 ms, 0.08 ms, 0.09 ms, 0.1 ms, 0.2 ms, 0.3 ms, 0.4 ms, 0.5 ms, 0.6 ms, 0.7 ms, 0.8 ms, 0.9 ms, 1 ms, 2 ms, 3 ms, 4 ms, 5 ms, 6 ms, 7 ms, 8 ms, 9 ms, 10 ms, 11 ms, 12 ms, 13 ms, 14 ms, 15 ms, 20 ms, 30 ms, 40 ms, 50 ms, 60 ms, 70 ms, 80 ms, 90 ms, 100 ms, 150 ms, 200 ms, 250 ms, 300 ms, 350 ms, 400 ms, 450 ms, 500 ms, 550 ms, 600 ms, 650 ms, 700 ms, 750 ms, 800 ms, 850 ms, 900 ms, 950 ms, or 1,000 ms.

In some cases, the number of applied voltage pulses delivered can be 1 or more, e.g., 2 or more, 3 or more, 4 or more, 5 or more, 6 or more, 7 or more, 8 or more, 9 or more, 10 or more, or 100 or more, e.g., 1-4, 2-5, 3-6, 4-7, 5-8, 6-9, 7-10, 8-11, 7-12, or 9-13, e.g., 0.01 to 1,000, e.g., from 1 to 10,1 to 50, 5 to 10, 5 to 15, 10 to 100, 25 to 200, 50 to 400, 100 to 600, 300 to 800, or 500 to 1,000, e.g., 1 to 100,1 to 50, or 1 to 10, e.g., about 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1,000, or more than 1000.

In some instances, the number of applied voltage pulses delivered can be 1 or more. For example, in some instances, the number of applied voltage pulses delivered is from 1,000 to 1,000,000, e.g., from 1,000 to 10,000 (e.g., from 1,000 to 2,000, from 2,000 to 3,000, from 3,000 to 4,000, from 4,000 to 5,000, from 5,000 to 6,000, from 6,000 to 7,000, from 7,000 to 8,000, from 8,000 to 9,000, or from 9,000 to 10,000, e.g., 1,000, 2,000, 3,000, 4,000, 5,000, 6,000, 7,000, 8,000, 9,000, or 10,000), from 10,000 to 100,000 (e.g., from 10,000 to 20,000, from 20,000 to 30,000, from 30,000 to 40,000, from 40,000 to 50,000, from 50,000 to 60,000, from 60,000 to 70,000, from 70,000 to 80,000, from 80,000 to 90,000, or from 90,000 to 100,000, e.g., 10,000, 25,000, 30,000, 40,000, 50,000, 60,000, 70,000, 75,000, 80,000, 90,000, or 100,000), or from 100,000 to 1,000,000 (e.g., from 100,000 to 200,000, from 200,000 to 300,000, from 300,000 to 400,000, from 400,000 to 500,000, from 500,000 to 600,000, from 600,000 to 700,000, from 700,000 to 800,000, from 800,000 to 900,000, or from 900,000 to 1,000,000, e.g., about 100,000, 200,000, 250,000, 300,000, 400,000, 500,000, 600,000, 700,000, 750,000, 800,000, 900,000, or 1,000,000).

The pulses of applied voltage can, in some instances, be delivered at a duty cycle of 1% to 100%, e.g., from 1% to 10%, 2.5% to 20%, 5% to 40%, 10% to 60%, 30% to 80%, or 50% to 100%, e.g., 0.01% to 100%, 0.1% to 99%, 1% to 97%, or 10% to 95%, e.g., about 1%, 2%, 3%, 4%, 5%, 6%, 7%, 8%, 9%, 10%, 11%, 12%, 15%, 20%, 25%, 30%, 35%, 40%, 45%, 50%, 55%, 60%, 65%, 70%, 75%, 80%, 85%, 90%, 95%, or 100%.

Device(s) of the invention, when the electrodes are connected to the source of electrical potential and energized, generate a localized electric field in the active zone that, in combination with mechanical energy (e.g., from the flow) transfect cells that pass through. In some cases, the electric field generated in the active zone has a magnitude from 2 V/cm to 50,000 V/cm, e.g., 2 V/cm to 1,000 V/cm, 100 V/cm to 1,000 V/cm, 100 V/cm to 5,000 V/cm, 400 V/cm to 2,000 V/cm, 400 to 1000 V/cm, 500 V/cm to 10,000 V/cm, 1000 V/cm to 25,000 V/cm, or from 5,000 V/cm to 50,000 V/cm, e.g., from 2 V/cm to 20,000 V/cm, 5 V/cm to 10,000 V/cm, or 100 V/cm to 1,000 V/cm, e.g., from about 2 V/cm, 3 V/cm, 4 V/cm, 5 V/cm, 6 V/cm, 7 V/cm, 8 V/cm, 9 V/cm, 10 V/cm, 20 V/cm, 30 V/cm, 40 V/cm, 50 V/cm, 60 V/cm, 70 V/cm, 80

V/cm, 90 V/cm, 100 V/cm, 200 V/cm, 300 V/cm, 400 V/cm, 500 V/cm, 600 V/cm, 700 V/cm, 800 V/cm, 900 V/cm, 1,000 V/cm, 2,000 V/cm, 3,000 V/cm, 4,000 V/cm, 5,000 V/cm, 6,000 V/cm, 7,000 V/cm, 8,000 V/cm, 9,000 V/cm, 10,000 V/cm, 15,000 V/cm, 20,000 V/cm, 25,000 V/cm, 30,000 V/cm, 35,000 V/cm, 40,000 V/cm, 45,000 V/cm, or 50,000 V/cm.

Systems of the invention typically include a fluid delivery source that is configured to deliver the plurality of cells suspended in the fluid through the entry zone to the active zone (e.g., through the first electrode) and out of the active zone (e.g., through the second electrode), e.g., to the recovery zone. Fluid delivery sources typically includes pumps, including, but not limited to, high pressure sources, syringe pumps, micropumps, or peristaltic pumps. Alternatively, fluids can be delivered by the displacement of a working fluid against a reservoir of the fluid to be delivered or by air displacement. Other fluid delivery sources are known in the art. In some cases, the fluid delivery source is configured to flow cells suspended in a fluid by the application of a positive pressure. Without wishing to be bound by any particular theory, the flow rate at which cells in a suspension are flowed through devices of the invention and the specific geometry of the active zone of devices of the invention will determine the residence time of the cells in the electric field in the active zone.

In some instances, the volumetric flow rate of fluid delivered from a fluid delivery source has a volumetric flow rate of 0.001 mL/min to 1,000 mL/min per active zone, e.g., from 0.001 mL/min to 0.1 mL/min, 0.01 mL/min to 1 mL/min, 0.1 mL/min to 10 mUmin, 1 mL/min to 50 mL/min, 10 mL/min to 100 mL/min, 25 mL/min to 200 mL/min, 50 mL/min to 400 mUmin, 100 mL/min to 600 mL/min, 300 mL/min to 800 mL/min, or 500 mL/min to 1,000 mL/min per active zone, e.g., about 0.001 mL/min, 0.002 mL/min, 0.003 mL/min, 0.004 mL/min, 0.005 mL/min, 0.006 mL/min, 0.007 mL/min, 0.008 mL/min, 0.009 mL/min, 0.01 mL/min, 0.02 mL/min, 0.03 mL/min, 0.04 mL/min, 0.05 mL/min, 0.06 mL/min, 0.07 mL/min, 0.08 mL/min, 0.09 mL/min, 0.1 mL/min, 0.2 mL/min, 0.3 mL/min, 0.4 mL/min, 0.5 mL/min, 0.6 mL/min, 0.7 mL/min, 0.8 mL/min, 0.9 mL/min, 1 mL/min, 2 mL/min, 3 mL/min, 4 mL/min, 5 mL/min, 6 mL/min, 7 mL/min, 8 mL/min, 9 mL/min, 10 mL/min, 15 mL/min, 20 mL/min, 25 mL/min, 30 mL/min, 35 mL/min, 40 mL/min, 45 mL/min, 50 mL/min, 55 mL/min, 60 mL/min, 65 mL/min, 70 mL/min, 75 mL/min, 80 mL/min, 85 mL/min, 90 mL/min, 95 mL/min, 100 mL/min, 150 mL/min, 200 mL/min, 250 mL/min, 300 mL/min, 350 mL/min, 400 mL/min, 450 mL/min, 500 mL/min, 550 mL/min, 600 mL/min, 650 mL/min, 700 mL/min, 750 mL/min, 800 mL/min, 850 mL/min, 900 mL/min, 950 mL/min, or 1,000 mL/min. In particular embodiments, the flow rate is from 10 mL/min to 100 mL/min per active zone, e.g., about 10 mL/min, 20 mL/min, 30 mL/min, 40 mL/min, 50 mL/min, 60 mL/min, 70 mL/min, 80 mL/min, 90 mL/min, or 100 mL/min per active zone.

In some instances, a Reynolds number of a liquid while passing through the active zone is between 10 and 3,000 (e.g., 10 to 100, 25 to 200, 50 to 400, 100 to 600 mL/min, 300 mL/min to 800 mL/min, 500 to 1,000, 800 to 1,500, 1,200 to 2,000, 1,800 to 2,500, or 2,400 to 3000, e.g., about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1,000, 1,500, 2,000, 2,050, or 3,000).

In some instances, a peak pressure of a liquid while passing through the active zone is between 1×10⁻³ Pa and 9.5×10⁴ Pa, e.g., between 0.001 to 9,500 (e.g., 0.001 Pa to 0.1 Pa, 0.01 Pa to 1 Pa, 0.1 Pa to 10 Pa, 1 Pa to 50 Pa, 10 Pa to 100 Pa, 25 Pa to 200 Pa, 50 Pa to 400 Pa, 100 Pa to 600 Pa, 300 Pa to 800 Pa, or 500 Pa to 1,000 Pa, 1,000 Pa to 6,000 Pa, 3,000 Pa to 8,000 Pa, 5,000 Pa to 9,000 Pa, or 7,500 Pa to 9,500 Pa, e.g., about 0.001 Pa, 0.002 Pa, 0.003 Pa, 0.004 Pa, 0.005 Pa, 0.006 Pa, 0.007

Pa, 0.008 Pa, 0.009 Pa, 0.01 Pa, 0.02 Pa, 0.03 Pa, 0.04 Pa, 0.05 Pa, 0.06 Pa, 0.07 Pa, 0.08 Pa, 0.09 Pa, 0.1 Pa, 0.2 Pa, 0.3 Pa, 0.4 Pa, 0.5 Pa, 0.6 Pa, 0.7 Pa, 0.8 Pa, 0.9 Pa, 1 Pa, 2 Pa, 3 Pa, 4 Pa, 5 Pa, 6 Pa, 7 Pa, 8 Pa, 9 Pa, 10 Pa, 15 Pa, 20 Pa, 25 Pa, 30 Pa, 35 Pa, 40 Pa, 45 Pa, 50 Pa, 55 Pa, 60 Pa, 65 Pa, 70 Pa, 75 Pa, 80 Pa, 85 Pa, 90 Pa, 95 Pa, 100 Pa, 150 Pa, 200 Pa, 250 Pa, 300 Pa, 350 Pa, 400 Pa, 450 Pa, 500 Pa, 550 Pa, 600 Pa, 650 Pa, 700 Pa, 750 Pa, 800 Pa, 850 Pa, 900 Pa, 950 Pa, 1,000 Pa, 1,100

Pa, 1,500 Pa, 2,000 Pa, 2,500 Pa, 3,000 Pa, 3,500 Pa, 4,000 Pa, 4,500 Pa, 5,000 Pa, 5,500 Pa, 6,000 Pa, 6,500 Pa, 7,000 Pa, 7,500 Pa, 8,000 Pa, 8,500 Pa, 9,000 Pa, or 9,500 Pa, or e.g., about 3,300 Pa (e.g., 2,500 to 4,000 Pa, e.g., 2,500 Pa to 3,000 Pa, 2,800 to 3,300 Pa, 3,100 Pa to 3,400 Pa), e.g., about 2,800 Pa, 2,900 Pa, 3,000 Pa, 3,100 Pa, 3,200 Pa, 3,300 Pa, 3,400 Pa, or 3,500 Pa. In some instances, an average flow velocity of a liquid while passing through the active zone is between 1×10⁻² m/s and 10 m/s, e.g., between 0.01 and 1 m/s (e.g., between 0.01 and 0.05 m/s, 0.05 and 0.1 m/s, 0.1 and 0.5 m/s, 0.5 and 1 m/s, 1.5 and 2 m/s, 1 and 2 m/s, 2 and 3 m/s, 3 and 4 m/s, 4 and 5 m/s, 5 and 6 m/s, 6 and 7 m/s, 7 and 8 m/s, 8 and 9 m/s, or 9 and 10 m/s), e.g., between 0.1 and 5 m/s, between 0.4 and 1.4 m/s, between 0.65 and 1.3 m/s, or between 0.26 and 2.08 m/s, e.g., about 0.1 m/s, 0.2 m/s, 0.3 m/s, 0.4 m/s, 0.5 m/s, 0.6 m/s, 0.7 m/s, 0.8 m/s, 0.9 m/s, 1.0 m/s, 1.1 m/s, 1.2 m/s, 1.3 m/s, 1.4 m/s, 1.5 m/s, 2 m/s, 3 m/s, 4 m/s, 5 m/s, 6 m/s, 7 m/s, 8 m/s, 9 m/s, or 10 m/s.

The residence time of cells in the active zone of devices of the invention may be from 0.5 ms to 50 ms, e.g., from 0.5 ms to 5 ms, 1 ms to 10 ms, 5 ms to 15 ms, 10 ms to 20 ms, 15 ms to 25 ms, 20 ms to 30 ms, 25 ms to 35 ms, 30 ms to 40 ms, 35 ms to 45 ms, or 40 ms to 50 ms, e.g., about 0.5 ms, 0.6 ms, 0.7 ms, 0.8 ms, 0.9 ms, 1 ms, 1.5 ms, 2 ms, 2.5 ms, 3 ms, 3.5 ms, 4 ms, 4.5 ms, 5 ms, 5.5 ms, 6 ms, 6.5 ms, 7 ms, 7.5 ms, 8 ms, 8.5 ms, 9 ms, 9.5 ms, 10 ms, 10.5 ms, 11 ms, 11.5 ms, 12 ms, 12.5 ms, 13 ms, 13.5 ms, 14 ms, 14.5 ms, 15 ms, 20 ms, 25 ms, 30 ms, 35 ms, 40 ms, 45 ms, or 50 ms. In some embodiments, the residence time is from 5-20 ms (e.g., from 6-18 ms, 8-15 ms, or 5-14 ms).

Systems of the invention typically feature a housing that contains and supports the device(s) of the invention and any necessary electrical connections, e.g., electrode connections. The housing may be configured to hold and energize a single device of the invention, or alternatively, may be configured to hold and simultaneously energize a plurality of devices of the invention. The housing may include a thermal controller that is able to regulate the temperature of the devices of the invention or thermally regulate a component of the system, e.g., a fluid, e.g., a buffer or suspension containing cells, during transfection. The thermal controller may be configured to heat the devices of the invention, or a component of a system thereof, cool the devices of the invention, or a component of a system thereof, or perform both operations. When configured to heat the devices of the invention, or a component of a system thereof, suitable thermal controllers include, but are not limited to, heating blocks or mantles, liquid heating, e.g., immersion or circulating fluid baths, battery operated heaters, or resistive heaters, e.g., thin film heaters, e.g., heat tape. When configured to cool the devices of the invention, or a component of a system thereof, suitable thermal controllers include, but are not limited to, liquid cooling, e.g., immersion or circulating fluid baths, evaporative coolers, or thermoelectric, e.g., Peltier coolers. For example, when implemented with liquid cooling, a device of the invention or a housing configured to hold devices of the invention may be in direct contact with tubing that circulates a chilled fluid or surrounded in a cooling jacket including tubing that circulates a chilled fluid. Other heating and cooling elements are known in the art.

In some embodiments, the housing (e.g., cartridge) is configured for use with and/or insertion into an automated closed system that is used to deliver cell therapies to patients in a clinical or hospital setting.

In some embodiments, the housing (e.g., cartridge) further includes a cooling/heating area/enclosure for cell suspension and/or buffer storage during, before and after electro-mechanical transfection of the specimen. In some embodiments, the system (e.g., device and housing) is externally powered.

In some embodiments, devices of the invention include a touchscreen user interface or other alternative user interface(s) that enables the user to select parameters such as flow rate, waveforms, applied potential, volume to transfect, time delay, cooling features, heating features, transfection status, progress and other parameters used to optimize the electro-mechanical transfection or electro-mechanical protocol. In some embodiments, the user interface also contains pre-formulated parameter selections that enable the user to operate the system at specific parameters and conditions that have previously been validated by user or as recommended by the manufacturers. In some embodiments, the user interface may be connected to programming that allows for automated running of the system and/or running an algorithm to optimize transfection for a given sample of a known cell type and payload combination. In some embodiments, the optimization algorithms have the ability to adjust electro-mechanical parameters independently or autonomously if the user selects this functionality. In some embodiments, the optimization algorithms allow for continuous adjustment of the parameters used in the electro-mechanical transfection process that may depend on the cell type, conductivity of cell suspensions, volume of cell suspensions, dynamic viscosity, lifetime of the transfection cartridge(s), the physical state of the suspension, or the state of the transfection device(s).

In some embodiments, the optimization algorithms have the ability to perform predictive analysis based on known input cell-type parameters and to adjust electro-mechanical parameters accordingly. Input parameters to be measured include, but are not limited to, suspension conductivity, suspension temperature, suspension dynamic viscosity, cell morphology, cell size, and cell impedance. In some embodiments, the optimization algorithms adjust electro-mechanical parameters based on electrical signals within any of the devices of the invention. In some embodiments, the optimization algorithms adjust electro-mechanical parameters based on detected flow parameters within any of the devices of the invention. In some embodiments, the optimization algorithms adjust transfection parameters based on unique dimensionless input parameters. In some embodiments, the optimization algorithms have the ability to adjust electro-mechanical transfection parameters based on unique multivariate combinations of parameters that are predictive of high viability results, high efficiency results, or matched viability and efficiency results.

Systems of the invention may include one or more outer structures that are configured to cover the electrodes of one or more devices of the invention, e.g., to reduce end user exposure to live electrical connections. Typically, an electro-mechanical system will include one outer structure that covers its electrodes and active zone. The outer structure may be a non-conductive material, e.g., a non-conductive polymer, that includes structural features for electro-mechanically engaging the parts of the device, e.g., the electrodes or active zone. The outer structure may include one or more recesses, cutouts, or similar openings within the structure to accommodate the device. The outer structure may be configured to be a component that can be removed from the device. For example, the outer structure may include two separate components connected by a hinge, e.g., a living hinge, such that it can be folded over the device of the invention. Alternatively, the outer structure may be one or more separate pieces that can be connected together using suitable mating features to form a single structure. In these embodiments, the outer structure may be affixed to the device of the invention using any suitable fastener, e.g., snaps, latches, button, or clips, which may be integrated into the outer structure or externally connected to the outer structure. Other suitable fastener types are known in the art. In some embodiments, the outer structure includes one or more alignment features, e.g., pins, divots, grooves, or tabs, that ensure correct alignment of the one or more pieces of the outer structure. In some cases, the outer structure is configured to be permanently connected to the devices of the invention.

In some embodiments, the housing (e.g., cartridge, e.g., outer structure) encapsulates one or more of the previously stated inventions or one or more devices used for continuous flow electro-mechanical transfection. In some embodiments, the housing (e.g., cartridge) is configured to allow use with and/or insertion into an automated closed system that delivers cell therapies to patients. In some embodiments, the housing further includes a cooling/heating area/enclosure for cell suspension and/or buffer storage during, before and after electro-mechanical transfection of the specimen. In some embodiments, the system (e.g., one or more devices and housing) is externally powered.

In some embodiments, the system also includes optimization algorithms that have the ability to adjust electro-mechanical parameters independently or autonomously if the user selects this functionality. These optimization algorithms allow for continuous adjustment of the parameters used in the transfection process that may depend on the cell type, conductivity, volume of suspensions, dynamic viscosity, lifetime of the electro-mechanical cartridge, the physical state of the suspension or the state of the electro-mechanical device.

In any of the embodiments of the outer structure described herein, the outer structure provides for electrical connection between an external source of electric potential and the electrodes of the devices of the invention. For example, the outer structure may include one or more electrical inputs for electrical connections, e.g., spades, banana plugs, or bayonet, e.g., BNC, connectors, that facilitate electrical connection between the source of electric potential and the electrodes of the devices of the invention inside the outer structure.

Devices and outer structures of the invention may be combined with additional external components, such as reagents, e.g., buffers, e.g., transfection or recovery buffers, and/or samples, in a kit. In some instances, a transfection buffer includes a composition appropriate for cell electro-mechanical transfection. In some instances, the transfection buffer includes a suitable concentration of one or more salts (e.g., potassium chloride, sodium chloride, potassium phosphate, potassium dihydrogen phosphate) or sugars (e.g., dextrose or myo-inositol), or any combination thereof, at a concentration from 0.1 to 200 mM (e.g., from 0.1 to 1.0 mM, from 1.0 mM to 10 mM, or from 10 mM to 100 mM).

### Buffers and Media

Devices and systems of the invention may be used with transfection buffer or with cell culture growth medium that contains additives to support transfection. Certain additives may be added to control the conductivity of transfection buffer and/or cell culture growth medium used, including KCl, MgCl₂, NaCl, glucose, Na₂HPO_{4,} NaH₂PO₄, Ca(NO₃)_{2,} mannitol, succinate, dextrose, hydroxyethyl piperazineethanesulfonic acid (HEPES), trehalose, CaCl₂, dimethyl sulfoxide (DMSO), K₂HPO₄, KH₂PO₄, ethylene-bis(oxyethylenenitrilo)tetraacetic acid (EGTA), KOH, NaOH, K₂SO₄, Na₂SO₄, histidine buffer, citrate buffer, phosphate-buffered saline (PBS), ATP-disodium salt, and NaHCO₃. Certain additives may be added to control the dynamic viscosity of transfection buffer and/or cell culture growth medium used, including Ficoll, dextran, polyethylene glycol (PEG), methylcellulose (MethoCel), collagen I, and Matrigel.

### Methods

The invention features methods of introducing a composition, e.g., genetic payload, into at least a portion of a plurality of cells suspended in a fluid, using the electro-mechanical transfection devices described herein. The methods described herein may be used to greatly increase the throughput of the delivery of compositions into cell types, often considered to be a bottleneck in the research fields of genetic engineering and therapeutic fields of gene-modified cell therapies. In particular, the methods described herein have significantly increased number of recovered cells, transfection efficiency and cell viability after transfection with applications to more cell types than typical methods of transfection, e.g., lentiviral transfection, or commercially available cell transfection instruments, e.g., electroporation-based instruments.

A composition is introduced into at least a portion of a plurality of cells suspended in a fluid by passing the fluid with the suspended cells, also containing the composition to be introduced into the cells, through a device of the invention, e.g., an electro-mechanical transfection device, as described herein. The composition and the cells suspended in the fluid can be delivered through the device of the invention by the application of a positive pressure, e.g., from a pump connected to a source of fluid, e.g., a peristaltic pump, a digital pipette, or automated liquid handling source. The composition and the cells suspended in the fluid pass from the entry zone to an active zone, e.g., disposed to pass through an electric field produced by two electrodes. As the composition and cells suspended in the fluid flow through the active zone, a potential difference is applied to the first and second electrodes, producing and thus exposing the cells to an electric field, which provides electrical energy to the cells, in the active zone. Cells in the fluid are simultaneously exposed to mechanical energy from the flow. The exposure of the cells to the generated electric field, in combination with the mechanical energy of the flow, enhances temporary permeability of the plurality of cells, thus introducing the composition into at least a portion of the plurality of cells. In particular embodiments, the electric field and flow are selected such that the dimensionless parameter,

$${Π}_{5} = \frac{ρσ {d}^{3}}{{μ}^{2}} ( \frac{{E}^{2}}{u} ) ,$$

has a value of between 1×10⁸ and 1×10¹⁰.

In some instances, the ratio of electrical energy provided to the flowing liquid by the electric field to mechanical energy provided by a pressure drop in the active zone is between 10³:1 to 10⁶:1, e.g., between 10³:1 and 10⁵:1, 10⁴:1 and 10⁶:1, 10³:1 and 10⁴:1, or 10⁵:1 and 10⁶:1 (e.g., about 10³:1, 10⁴:1, 10⁵:1, or 10⁶:1).

In some instances, methods of the invention involve first passing a test portion (e.g., a test portion from a larger plurality of cells) of the plurality of cells and a test composition through the active zone according to any method described herein. One or more test portions can be used to determine the optimal range of π₅, e.g., to find a range of π₅ which corresponds to a maximum cell viability, transfection efficiency, and/or engineered cell yield. The test portion (e.g., one having certain ratio of cells to composition) may be passed through the active zone at an average flow velocity (u) while applying an electric field (E), varying one or more of (u), (E), the liquid conductivity (σ), the liquid dynamic viscosity (μ), and the liquid density (ρ), to find a range of π₅ which corresponds to a maximum cell viability, transfection efficiency, and/or engineered cell yield. This may be repeated several (e.g., 2, 3, 4, 5, 6, 7, 8, 9, or 10 or more times) with the same ratio of cells to composition and/or different ratios.

Alternatively, or in addition, the test may be repeated in an active zone having a different hydraulic diameter. After determining the appropriate range or ranges of π₅, the plurality of cells may be passed through the active zone (of appropriate hydraulic diameter) with the combination of (u), (E), (σ), (μ), and (ρ) to introduce the composition into the plurality of cells. Any one or any combination of the variables of (u), (E), (a), (p), and (p), may be varied.

In particular instances, the test portion is passed through the active zone with varying average flow velocity (u)while applying a constant electric field (E) with the electrodes, or average flow velocity may be at a constant velocity while the electric field is varied (e.g., by varying the voltage between the electrodes). One or both of these steps may be repeated one or more times, e.g., 2, 3, 4, 5, 6, 7, 8, 9, or 10 or more times.

In some instances of the methods, phenotypic markers of the cells associated with cell health, or the expression of certain surface markers, or certain cell properties, e.g., those required for therapeutic function, may not be altered relative to a baseline measurement of the cell phenotypic markers, or other measure of cell health, function, etc., upon exiting the active zone of devices of the invention. In particular instances, the plurality of cells has no measurable change in certain phenotypic markers associated with cell health or desired function (e.g., expression) upon exiting the active zone. For example, a baseline or control measurement to establish the cell phenotype may be the measurement of the expression of a cell surface marker on cells that have not been transfected using devices of the invention. A corresponding identical measurement of the expression of the same cell marker on cells that have been transfected using devices of the invention can be used to assess changes in cell phenotype. The cell phenotype is assessed via flow cytometry analysis of cell surface marker expression to ensure that the cell phenotype is minimally changed or unchanged after electro-mechanical transfection. Examples of the cell surface markers to evaluate include, but are not limited to, CD3, CD4, CD8, CD19, CD45RA, CD45RO, CD28, CD44, CD69, CD80, CD86, CD206, IL-2 receptor, CTLA4, OX40, PD-1, and TIM3, CD56, TNFa, IFNg, LAG3, TCR alpha/beta, CD64, SIRP alpha/beta (CD172a/b), Nestin, CD325 (N-Cadherin), CD183 (CXCR3), CD1 84 (CXCR4), CD197 (CCR7), CD7, CD11b, CCR7 (CD197), CD16, CD56, TIGIT, TRA-1-60, Nanog, TCR gamma/delta, OCT4, T-bet, GATA-3, FoxP3, IL-17, B220, CD25, IgM, PD-L1, IL-23, IL-12, CD11c, and F4/80. Cell morphology is assessed (e.g., using bright field or fluorescent microscopy) to confirm lack of phenotypic changes after electro-mechanical transfection.

In some instances, after introduction of the composition into at least a portion of the plurality of cells, the plurality of cells are stored in a recovery buffer. The recovery buffer is configured to promote the final closing of the pores that were formed in the plurality of cells. Recovery buffers typically include cell culture media that may include other ingredients for cell nourishment and growth, e.g., serum, minerals, etc. A skilled artisan can appreciate that the choice of recovery buffer will depend on the cell type undergoing electro-mechanical transfection.

Cell flux, i.e., number of cells processed per minute per active zone, e.g., number of cells transfected per minute, typically ranges from 10³ cells/min to 10¹¹ cells/min, e.g., 10³ cells/min to 10⁴ cells/min, 5×10³ cells/min to 5×10⁴ cells/min, 10⁵ cells/min to 10⁵ cells/min, 5×10⁵ cells/min to 5×10⁶ cells/min, 10⁶ cells/min to 10⁷ cells/min, 5×10⁶ cells/min to 5×10⁷ cells/min, 10⁷ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, or 10¹⁰ cells/min to 10¹¹ cells/min, e.g., about 10³ cells/min, 5×10³ cells/min, 10⁴ cells/min, 5×10⁴ cells/min, 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹⁰ cells/min, or 10¹¹ cells/min. In some instances of the methods, the composition is introduced into the plurality of cells at a flux of at least 1×10⁵ cells per minute per active zone, e.g., 10⁵ cells/min to 10⁵ cells/min, 5×10⁵ cells/min to 5×10⁶ cells/min, 10⁶ cells/min to 10⁷ cells/min, 5×10⁶ cells/min to 5×10⁷ cells/min, 10⁷ cells/min to 10⁸ cells/min, 5×10⁷ cells/min to 5×10⁸ cells/min, 10⁸ cells/min to 10⁹ cells/min, 5×10⁸ cells/min to 5×10⁹ cells/min, 10⁹ cells/min to 10⁹ cells/min, 5×10⁹ cells/min to 5×10¹⁰ cells/min, 10¹⁰ cells/min to 10¹¹ cells/min, or 10¹¹ cells/min to 10¹² cells/min, e.g., about 10³ cells/min, 5×10³ cells/min, 10⁴ cells/min, 5×10⁴ cells/min, 10⁵ cells/min, 5×10⁵ cells/min, 10⁶ cells/min, 5×10⁶ cells/min, 10⁷ cells/min, 5×10⁷ cells/min, 10⁸ cells/min, 5×10⁸ cells/min, 10⁹ cells/min, 5×10⁹ cells/min, 10¹⁰ cells/min, 5×10¹⁰ cells/min, 10¹¹ cells/min, or 10¹² cells/min.

In some embodiments of the method described herein, the volume of fluid with the suspended cells (e.g., displacement volume) and the composition to be introduced to the cells that are flowed through the active zone of devices of the invention may be from 0.001 mL to 2000 mL per active zone, 0.001 mL to 1000 mL, e.g., 0.001 mL to 1000 mL, e.g., from 0.001 mL to 0.1 mL, 0.01 mL to 1 mL, 0.01 mL to 750 mL, 0.01 mL to 1500 mL, 0.1 mL to 5 mL, 0.1 mL to 500 mL, 0.1 mL to 2000 mL, 1 mL to 10 mL, 1 mL to 1000 mL, 2 mL to 2000 mL, 2.5 mL to 20 mL, 5 mL to 40 mL, 10 mL to 60 mL, 10 mL to 1000 mL, 20 mL to 2000 mL, 30 mL to 80 mL, 50 mL to 200 mL, 100 mL to 500 mL, or 250 mL to 750 mL, 500 mL to 1000 mL, 500 mL to 2000 mL, 750 mL to 1500 mL, or 1000 mL to 2000 mL, e.g., 0.01 mL to 100 mL, 0.1 mL to 99 mL, 1 mL to 97 mL, or 10 mL to 95 mL, e.g., 0.0025 mL to 10 mL, 0.01 mL to 1 mL, or 0.025 mL to 0.1 mL, e.g., about 0.001 mL, 0.0025 mL, 0.005 mL, 0.0075 mL, 0.01 mL, 0.025 mL, 0.05 mL, 0.075 mL, 0.1 mL, 0.25 mL, 0.5 mL, 0.75 mL, 1 mL, 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, 550 mL, 600 mL, 650 mL, 700 mL, 750 mL, 800 mL, 850 mL, 900 mL, 950 mL, 1000 mL, 1050 mL, 1100 mL, 1150 mL, 1200 mL, 1250 mL, 1300 mL, 1350 mL, 1400 mL, 1450 mL, 1500 mL, 1550 mL, 1600 mL, 1650 mL, 1700 mL, 1750 mL, 1800 mL, 1850 mL, 1900 mL, 1950 mL, or 2000 mL.

In some embodiments, the volume of fluid that is flowed through the active zone of devices of the invention (e.g., displacement volume), displacement rate, or other controlled parameters may or may not affect the transfection efficiency of a plurality of cells. In some embodiments, the devices of the invention are configured for use with an automated fluid handling platform that can process a plurality of cells in volumes of approximately 10-200 μL per reaction. In some embodiments, the devices of the invention are part of a system that can process volumes up to multiple liters per reaction. In some embodiments, the automated fluid handling platform is configured for use with one or more fluid delivery sources (e.g., pumps, e.g., syringe pumps, micropumps, or peristaltic pumps) that deliver the volume of fluid that is flowed through the active zone of devices of the invention. In some embodiments, the volume of fluid that is flowed through the active zone of devices of the invention can be delivered by the displacement of a working fluid against a reservoir of the fluid to be delivered or by air displacement. In some embodiments, the fluid delivery source is configured to flow cells suspended in a fluid by the application of a positive pressure.

In certain aspects, the electrical conductivity of the fluid where the cells are suspended can affect the electro-mechanical transfection of the cells in the suspension. The conductivity of the fluid with the suspended cells may be from 0.001 mS to 500 mS, e.g., from 0.001 mS to 0.1 mS, 0.01 mS to 1 mS, 0.1 mS to 10 mS, 1 mS to 50 mS, 10 mS to 100 mS, 25 mS to 200 mS, 50 mS to 400 mS, or 100 mS to 500 mS, e.g., 0.01 mS to 100 mS, 0.1 mS to 50 mS, or 1 to 20 mS, e.g., about 0.001 mS, 0.002 mS, 0.003 mS, 0.004 mS, 0.005 mS, 0.006 mS, 0.007 mS, 0.008 mS, 0.009 mS, 0.01 mS, 0.02 mS, 0.03 mS, 0.04 mS, 0.05 mS, 0.06 mS, 0.07 mS, 0.08 mS, 0.09 mS, 0.1 mS, 0.2 mS, 0.3 mS, 0.4 mS, 0.5 mS, 0.6 mS, 0.7 mS, 0.8 mS, 0.9 mS, 1 mS, 2 mS, 3 mS, 4 mS, 5 mS, 6 mS, 7 mS, 8 mS, 9 mS, 10 mS, 15 mS, 20 mS, 25 mS, 30 mS, 35 mS, 40 mS, 45 mS, 50 mS, 55 mS, 60 mS, 65 mS, 70 mS, 75 mS, 80 mS, 85 mS, 90 mS, 95 mS, 100 mS, 150 mS, 200 mS, 250 mS, 300 mS, 350 mS, 400 mS, 450 mS, or 500 mS.

Methods of the invention can deliver compositions to a variety of cell types including, but not limited to, mammalian cells, eukaryotes, prokaryotes, synthetic cells, human cells, animal cells, plant cells, primary cells, cell lines, suspension cells, adherent cells, unstimulated cells, stimulated cells, or activated cells immune cells, solid tumor cells, stem cells (e.g., primary human induced pluripotent stem cells, e.g., iPSCs, embryonic stem cells, e.g., ESCs, mesenchymal stem cells, e.g., MSCs, or hematopoietic stem cells, e.g., HSCs), blood cells (e.g., red blood cells), T cells (e.g., primary human T cells), B cells, antigen presenting cells (APCs), natural killer (NK) cells (e.g., primary human NK cells), monocytes (e.g., primary human monocytes), macrophages (e.g., primary human macrophages), and peripheral blood mononuclear cells (PBMCs), neutrophils, dendritic cells, human embryonic kidney (e.g., HEK-293) cells, or Chinese hamster ovary (e.g., CHO-K1) cells. Typical cell numbers that can be transfected may be from 10⁴ cells to 10¹² cells per active zone, (e.g., 10⁴ cells to 10⁵ cells, 10⁴ cells to 10⁶ cells, 10⁴ cells to 10⁷ cells, 5×10⁴ cells to 5×10⁵ cells, 10⁵ cells to 10⁶ cells, 10⁵ cells to 10⁷ cells, 2.5×10⁵ cells to 10⁶ cells, 5×10⁵ cells to 5×10⁶ cells, 10⁶ cells to 10⁷ cells, 10⁶ cells to 10⁸ cells, 10⁶ cells to 10¹² cells, 5×10⁶ cells to 5×10⁷ cells, 10⁷ cells to 10⁸ cells, 10⁷ cells to 10⁹ cells, 10⁷ cells to 10¹² cells, 5×10⁷ cells to 5×10⁸ cells, 10⁸ cells to 10⁹ cells, 10⁸ cells to 10¹⁰ cells, 10⁸ cells to 10¹² cells, 5×10⁸ cells to 5×10⁹ cells, 10⁹ cells to 10¹⁰ cells, 10⁹ cells to 10¹¹ cells, 10¹⁰ cells to 10¹¹ cells, 10¹⁰ cells to 10¹² cells, or 10¹¹ cells to 10¹² cells, e.g., about 10⁴ cells, 2.5×10⁴ cells, 5×10⁴ cells, 10⁵ cells, 2.5×10⁵ cells, 5×10⁵ cells, 10⁶ cells, 2.5×10⁶ cells, 5×10⁶ cells, 10⁷ cells, 2.5×10⁷ cells, 5×10⁷ cells, 10⁸ cells, 2.5×10⁸ cells, 5×10⁸ cells, 10⁹ cells, 2.5×10⁹ cells, 5×10⁹ cells, 10¹⁰ cells, 5×10¹⁰ cells, 10¹¹ cells, or 10¹² cells).

Methods of the invention described herein may deliver a variety of compositions to the cells suspended in the fluid. Compositions that can be delivered to the cells include, but are not limited to, therapeutic agents, vitamins, nanoparticles, charged molecules, e.g., ions in solution, uncharged molecules, nucleic acids, e.g., DNA or RNA, CRISPR-Cas complex, proteins, polymers, a ribonucleoprotein (RNP), engineered nucleases, transcription activator-like effector nucleases (TALENs), zinc-finger nucleases (ZFNs), homing nucleases, meganucleases (MNs), megaTALs, enzymes, peptides, transposons, or polysaccharides, e.g., dextran, e.g., dextran sulfate. Exemplary compositions that can be delivered to cells in a suspension include nucleic acids, oligonucleotides, antibodies (or an antibody fragment, e.g., a bispecific fragment, a trispecific fragment, Fab, F(ab′)2, or a single-chain variable fragment (scFv)), amino acids, peptides, proteins, gene therapeutics, genome engineering therapeutics, epigenome engineering therapeutics, carbohydrates, chemical drugs, contrast agents, magnetic particles, polymer beads, metal nanoparticles, metal microparticles, quantum dots, antioxidants, antibiotic agents, hormones, nucleoproteins, polysaccharides, glycoproteins, lipoproteins, steroids, anti-inflammatory agents, anti-microbial agents, chemotherapeutic agents, exosomes, outer membrane vesicles, vaccines, viruses, bacteriophages, adjuvants, minerals, and combinations thereof. A composition to be delivered may include a single compound, such as the compounds described herein. Alternatively, the composition to be delivered may include a plurality of compounds or components targeting different genes.

Typical concentrations of the composition in the fluid may be from 0.0001 μg/mL to 1000 μg/mL, (e.g., from 0.0001 μg/mL to 0.001 μg/mL, 0.001 μg/mL to 0.01 μg/mL, 0.001 μg/mL to 5 μg/mL, 0.005 μg/mL to 0.1 μg/mL, 0.01 μg/mL to 0.1 μg/mL, 0.01 μg/mL to 1 μg/mL, 0.1 μg/mL to 1 μg/mL, 0.1 μg/mL to 5 μg/mL, 1 μg/mL to 10 μg/mL, 1 μg/mL to 50 μg/mL, 1 μg/mL to 100 μg/mL, 2.5 μg/mL to 15 μg/mL, 5 μg/mL to 25 μg/mL, 5 μg/mL to 50 μg/mL, 5 μg/mL to 500 μg/mL, 7.5 μg/mL to 75 μg/mL, 10 μg/mL to 100 μg/mL, 10 μg/mL to 1,000 μg/mL, 25 μg/mL to 50 μg/mL, 25 μg/mL to 250 μg/mL, 25 μg/mL to 500 μg/mL, 50 μg/mL to 100 μg/mL, 50 μg/mL to 250 μg/mL, 50 μg/mL to 750 μg/mL, 100 μg/mL to 300 μg/mL, 100 μg/mL to 1,000 μg/mL, 200 μg/mL to 400 μg/mL, 250 μg/mL to 500 μg/mL, 350 μg/mL to 500 μg/mL, 400 μg/mL to 1,000 μg/mL, 500 μg/mL to 750 μg/mL, 650 μg/mL to 1,000 μg/mL, or 800 μg/mL to 1,000 μg/mL, e.g., about 0.0001 μg/mL, 0.0005 μg/mL, 0.001 μg/mL, 0.005 μg/mL, 0.01 μg/mL, 0.02 μg/mL, 0.03 μg/mL, 0.04 μg/mL, 0.05 μg/mL, 0.06 μg/mL, 0.07 μg/mL, 0.08 μg/mL, 0.09 μg/mL, 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.5 μg/mL, 2 μg/mL, 2.5 μg/mL, 3 μg/mL, 3.5 μg/mL, 4 μg/mL, 4.5 μg/mL, 5 μg/mL, 5.5 μg/mL, 6 μg/mL, 6.5 μg/mL, 7 μg/mL, 7.5 μg/mL, 8 μg/mL, 8.5 μg/mL, 9 μg/mL, 9.5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL, or 1,000 μg/mL).

Typical concentrations of the composition in the fluid may be between 0.0001 μM and 20 μM (e.g., from 0.0001 μM to 0.001 μM, 0.001 μM to 0.01 μM, 0.001 μM to 5 μM, 0.005 μM to 0.1 μM, 0.01 μM to 0.1 μM, 0.01 μM to 1 μM, 0.1 μM to 1 μM, 0.1 μM to 5 μM, 1 μM to 10 μM, 1 μM to 15 μM, or 1 μM to 20 μM, e.g., about 0.0001 μM, 0.0005 μM, 0.001 μM, 0.005 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 11 μM, 12 μM, 13 μM, 14 μM, 15 μM, 16 μM, 17 μM, 18 μM, 19 μM, or 20 μM).

In some cases, the temperature of the fluid with the suspended cells and the composition is controlled using a thermal controller that is incorporated into a housing that supports the device(s) of the invention. The temperature of the fluid is controlled to reduce the effects of Joule heating originating from the electric field generated in the active zone, as too high a temperature may compromise cell viability post-electro-mechanical transfection. The temperature of the fluid may be from 0° C. to 40° C., e.g., from 0° C. to 10° C., 1° C. to 5° C., 2° C. to 15° C., 3° C. to 20° C., 4° C. to 25° C., 5° C. to 30° C., 7° C. to 35° C., 9° C. to 40° C., 10° C. to 38° C., 15° C. to 40° C., 20° C. to 40° C., 25° C. to 40° C., or 35° C. to 40° C., e.g., about 0° C., 1° C., 2° C., 3° C., 4° C., 5° C., 6° C., 7° C., 8° C., 9° C., 10° C., 11° C. 12° C., 13° C., 14° C., 15° C., 16° C., 17° C., 18° C., 19° C., 20° C., 21° C. 22° C., 23° C., 24° C., 25° C., 26° C., 27° C., 28° C., 29° C., 30° C. 31° C., 32° C., 33° C., 34° C., 35° C., 36° C., 37° C., 38° C., 39° C., or 40° C.

Cells transfected using the methods of the invention are more efficiently transfected and have higher viability than using typical methods of transfection, e.g., lentiviral transfection, or commercially available cell transfection instruments. For example, the transfection efficiency, i.e., the efficiency of successfully delivering a composition to a cell, for the methods described herein, may be from 0.1% to 99.9%, e.g., from 0.1% to 5%, 1% to 10%, 2.5% to 20%, 5% to 40%, 10% to 60%, 30% to 80%, or 50% to 99.9%, e.g., from 10% to 90%, from 25% to 85%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99.9%.

The cell viability, i.e., the number or percentage of healthy cells following an electro-mechanical transfection process, of the cells suspended in the fluid after having a composition introduced using methods of the invention described herein may be from 0.1% to 99.9%, e.g., from 0.1% to 5%, 1% to 10%, 2.5% to 20%, 5% to 40%, 10% to 60%, 30% to 80%, or 50% to 99.9%, e.g., from 10% to 90%, from 25% to 85%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99.9%.

The recovery yield, i.e., the percentage of live engineered cells collected after electro-mechanical transfection, may be from 0.1% to 500% at 24 h post-transfection, e.g., from 0.1% to 5%, 1% to 10%, 2.5% to 20%, 5% to 40%, 10% to 60%, 30% to 80%, 50% to 99.9%, from 75% to 150%, from 100% to 200%, from 150% to 250%, from 200% to 300%, from 250% to 350%, from 300% to 400%, from 350% to 450%, or from 400% to 500%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99.9%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, or 500%.

In some embodiments, the method produces a cell recovery yield, i.e., the percentage of live engineered cells collected after electro-mechanical transfection, of between 0.1% and 100% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 75% and 100%, between 85% and 100%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%).

A skilled artisan will appreciate that optimal conditions may vary depending on cell type or other factors. For each new cell type, the following parameters can be adjusted as necessary: waveform, electric field, pulse duration, buffer exposure time, buffer temperatures, and post-processing conditions.

### EXAMPLES

### Example 1—Application of Electro-mechanical Transfection

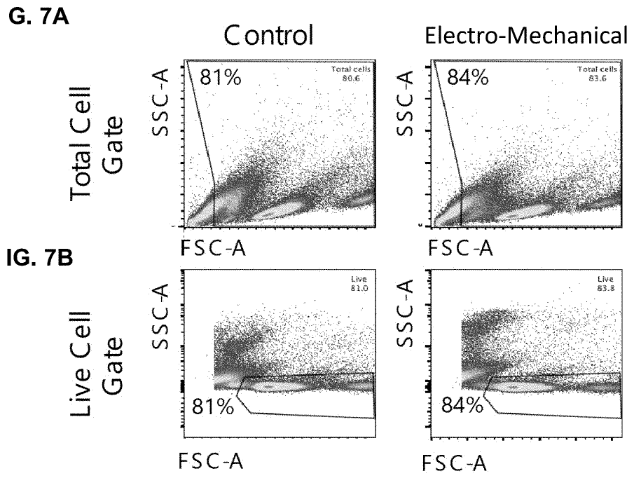

Electro-mechanical transfection implemented with an automated liquid handler is demonstrated herein. In this example, a flow cell was designed to integrate with the liquid handling system in order to enable delivery of electrical and mechanical energy to a cell suspension (FIG. 1). These flow cells include a pipette tip designed with a reservoir, enabling pickup and dispensing of cells and payload suspended in fluid buffer material. As the cells and payload suspended in the fluid buffer passed through the flow cell with a defined flow rate, a precise electric field is delivered across the flow cell via contact with electrodes placed across the flow cell region. These cells are dispensed into a 96 well plate containing growth media and cultured for 24 hours. Biological analysis is then performed to determine output metrics via flow cytometer (gating example can be found in FIG. 7).

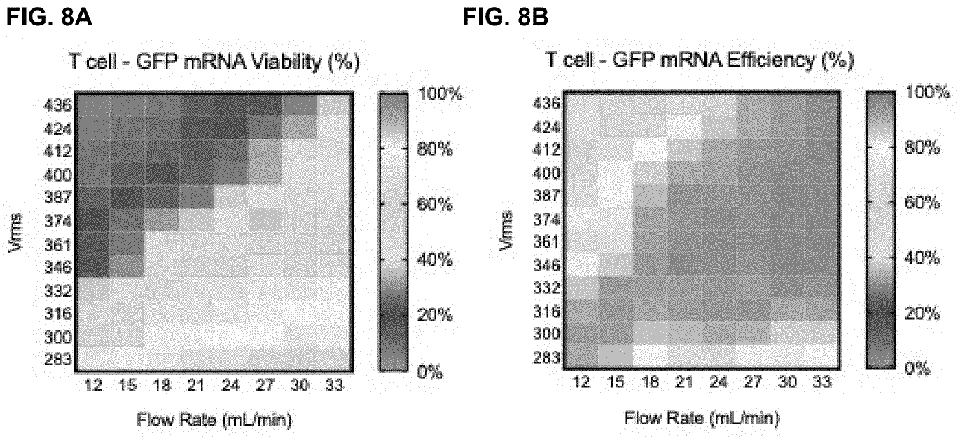

Effective use of electro-mechanical transfection requires determination of optimal conditions and parameters for the targeted cells and payload combination. These conditions and parameters include flow rate and electric field properties. In order to determine optimal conditions for the application of the electro-mechanical transfection for delivery of an mRNA-based payload into primary T cells, a plate-based matrix experiment was performed on a fully automated platform including an array of electro-mechanical devices integrated with a commercial liquid handling system (PerkinElmer JANUS® G3 system, Waltham, Mass.). Utilizing this technology, up to 96 independently programmed combinations of electro-mechanical transfection parameters can be delivered in a batchwise manner. In this set of experiments a reporter mRNA payload was delivered to day nine expanded human T cells 24 hours out of thaw. Primary human T cells were expanded using soluble anti-CD3 and anti-CD28 antibodies and resuspended in transfection buffer. The samples were recovered for culture and downstream analysis immediately after processing. Cell viability, live cell count, and transfection efficiency were measured via flow cytometry (Thermo Fisher Attune™ NxT); GFP (green fluorescent protein) mRNA was used to measure delivery efficiency to the expanded T cells (FIG. 8). This set of experiments resulted in multiple conditions (total 11) wherein transfected cells exhibit both high viability (greater than 70% live cells) and high transfection efficiency (greater than 90% GFP⁺).

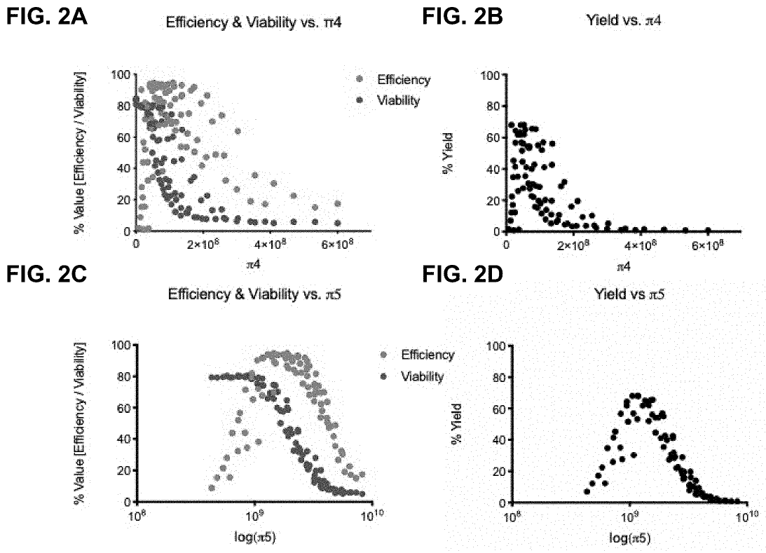

The most relevant cell transfection parameters, such as viability, transfection efficiency, and cell yield are expected to depend upon the dimensionless parameters defined above including π₁, π₂, Re, π₄ and combinations thereof (FIG. 2). A fifth dimensionless group, which is a combination of all four seems to govern the dominant physics associated with cell transfection. We will denote this dimensionless group as

$${Π}_{5} = \frac{( Re ) {Π}_{4}}{{Π}_{1}^{2}} = \frac{ρ σ {d}^{3}}{{μ}^{2}} ( \frac{{E}^{2}}{u} ) .$$

For a given cell type, in a particular transfection media, all of the terms outside of the parentheses can be considered a constant. Therefore, we expect that cell transfection will typically vary most significantly with the applied electric field, E (e.g., electrical energy), and the average velocity in the channel, u (e.g., mechanical energy). As shown in the examples below, we find that cell viability, transfection efficiency, and cell yield, all appear to have a strong dependence on π₅. This data provides evidence that for a fixed channel geometry, media, and cell type, the value of π₅ is one of the principal factors determining cell transfection results. This factor, which couples the effects of both mechanical and electrical energy inputs, further indicates that electro-mechanical transfection is physically distinct from purely electrical or purely mechanical methods of cell transfection.

### Example 2—Transcriptional profiling

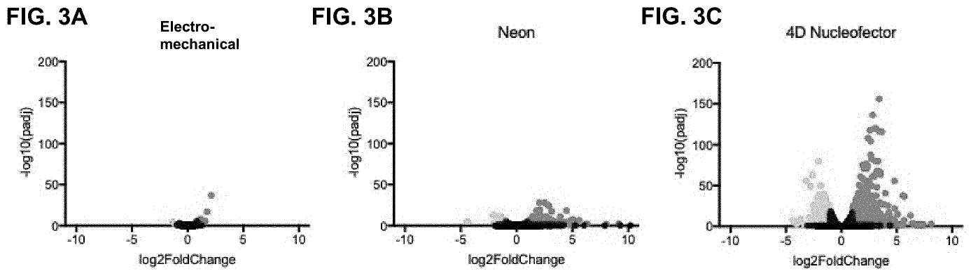

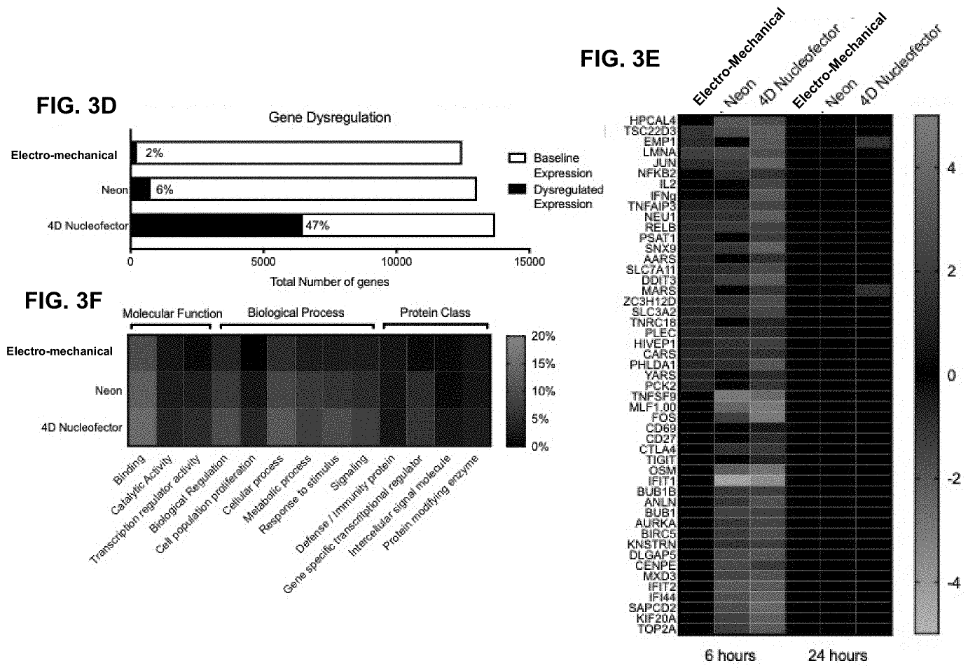

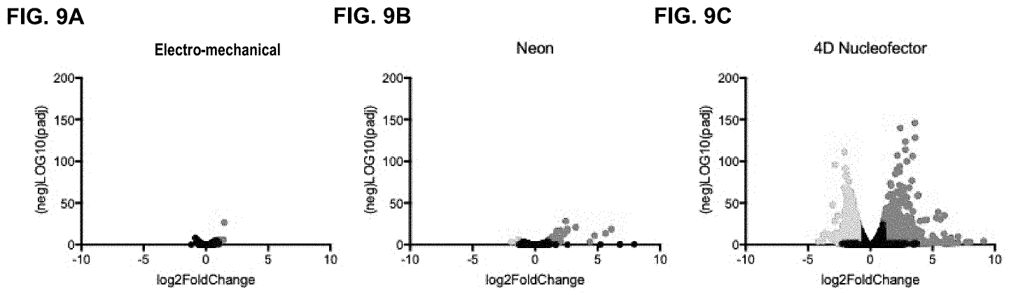

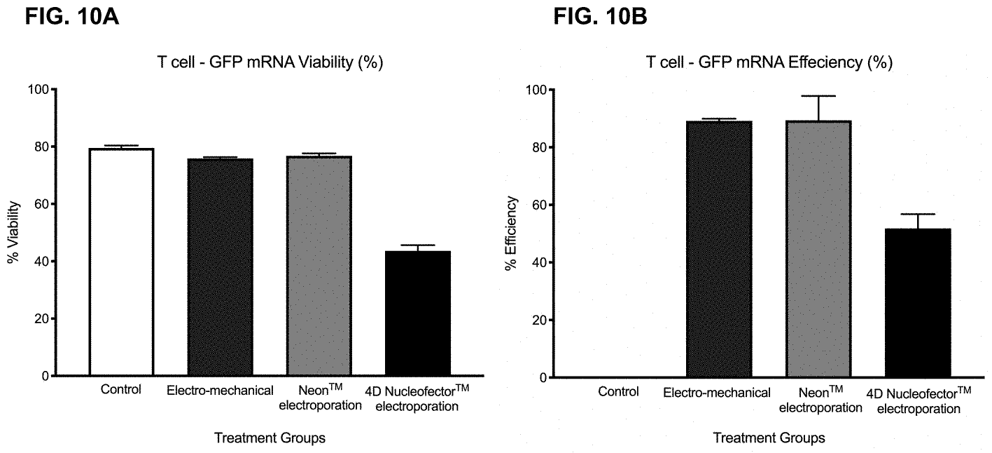

To further asses the effects of delivery of genetic payloads into T cells using electro-mechanical transfection, transcriptome analysis was performed to evaluate transcriptional changes that occur after processing (FIGS. 3A-3F). Additionally, commercially available non-viral electroporation-based systems were included for comparison metrics: the Neon™ transfection system from Thermo Fisher (referred to as ‘Neon™’) and the 4D Nucleofector™ from Lonza (referred to as ‘4D Nucleofector™’). Each system was evaluated using 100 μL reactions containing 5 M cells and device-specific proprietary programs and buffers. The program information for each device is provided in the Materials section. For each device, cells were processed without payload present and compared to a donor control that did not experience any processing. For this analysis, cells from two donors were processed in duplicate on each device. Significant (p<0.05) gene dysregulation, greater than 1-fold, at 6 hours after cell processing are shown in volcano plots as red (upregulated genes) or green (downregulated genes) in FIGS. 3A-3C (replicate data from the second donor is shown in FIG. 9). The electro-mechanical system exhibited a nearly baseline gene expression profile at 6 hours, with only 2% of all genes dysregulated by the electro-mechanical transfection process (FIG. 3D). The Neon™ static transfection system exhibited a low dysregulation profile at 6 hours, with only 6% of all genes dysregulated by this electroporation-based process (FIG. 3D). The 4D Nucleofector™ exhibited significant dysregulation at 6 hours, with 47% of all genes dysregulated by this electroporation-based transfection process (FIG. 3D).

The functional capability of a cell product can directly impact the effectiveness of the cells to drive the desired immunological response. For instance, it has been shown that edited cell differentiation and exhaustion can be linked to limited efficiency of T cell therapies. To better understand the impact of cell processing on T cell function, upregulation of genes commonly associated with T cell function were assessed at 6-hour and 24-hour time points (FIG. 3E). We included the proinflammatory cytokines (IFNγ and IL-2) and the activation receptors (CD69 and CD27) which were selected to assess process driven activation of the cells. Additionally, the exhaustion receptors (CTLA4 and TIGIT) were selected as indicators of process impact to cellular function downstream in treated cells. No upregulation of proinflammatory cytokines, activation receptors, or exhaustion markers were observed in electro-mechanical treated cells (FIG. 3E). Conversely, electroporation induced upregulation of these genes; the Neon™ transfection system upregulated CTLA4 and the 4D Nucleofector™ upregulated proinflammatory cytokines, activation receptors, and exhaustion markers in treated cells (FIG. 3E).

To further explore the impact to overall cell health and function gene ontology focused on molecular function, biological process, and protein class were assessed using the Protein Analysis Through Evolutionary Relationships (PANTHER) classification system. At the 6-hour time point electro-mechanical transfection showed that 6% of the total dysregulation was associated with protein class, 13% was attributed to molecular function, and 18% of the total dysregulation was associated with biological processes (FIG. 3F). For the Neon™ electroporation system, 10% of the total dysregulation was associated with protein class, 19% was attributed to molecular function, and 36% of the total dysregulation was associated with biological processes (FIG. 3F). Transfection with the 4D Nucleofector™ resulted in 13% of the total dysregulation being associated with protein class, 24% was attributed to molecular function, and 52% of the total dysregulation was associated with biological processes (FIG. 3F).

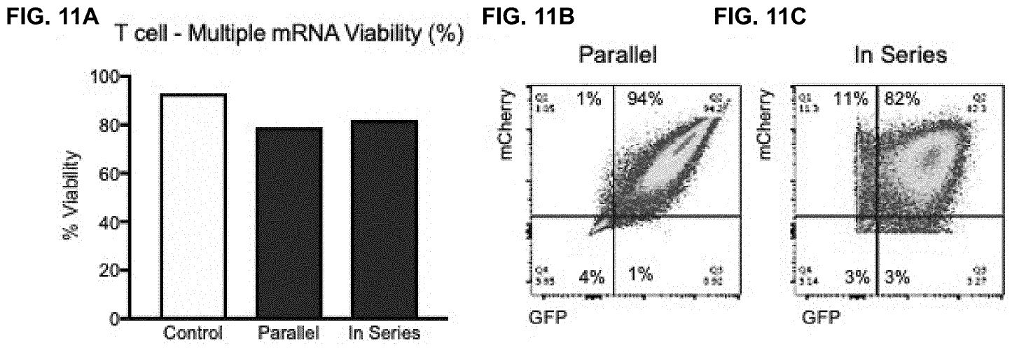

To correlate the transcriptome data with post-processing viability and delivery efficiency, cells from the same donors were transfected with reporter mRNA payload using the same programs and conditions for the electro-mechanical system, Neon™, and 4D Nucleofector™ platforms (FIG. 11A-11C). The electro-mechanical system exhibited high transfected cell viability of ˜80%, similar to the Neon™ system, while the 4D Nucleofector™ system exhibited low transfected cell viability ˜45% (FIG. 11A). Regarding delivery, both the electro-mechanical and Neon™ systems achieved high delivery efficiency of ˜90%, while the 4D Nucleofector™ system resulted in moderate delivery efficiency ˜50%. (FIG. 11B).

Taken together with the transcriptome analysis it is clear that non-viral delivery efficiency is not tied to poor cell product health post processing. Moreover, electro-mechanical transfection compares favorably to existing electroporation-based transfection devices in terms of all metrics, including gene dysregulation, viability, efficiency, and cell health outputs.

### Example 3—Delivery of Multiple mRNAs to Primary Human T Cells

We performed experiments to evaluate the capability of electro-mechanical transfection methods to deliver multiple payloads into a single cell both in parallel (i.e., co-delivery via a single treatment) and in series (i.e., staggered treatments, 48 hours apart). The parallel condition was performed on the same day with a cell mix containing two mRNAs, including a GFP reporter mRNA and a mCherry reporter mRNA, while the series treatments were performed two days apart with cell mixes containing a single mRNA at each time point, first with GFP reporter mRNA then mCherry mRNA 48 hours later. The mRNAs expressed fluorescent reporter genes to track delivery efficiency at the single cell level (FIGS. 11A-11C). The viability of primary T cells 24 hours after treatment with electro-mechanical transfection was −80% for both methods (FIG. 11A), demonstrating that parallel and in series transfections were not detrimental to cell health, allowing for repeat staggered transfection without significant loss in cell viability utilizing electro-mechanical technology. However, different expression profiles were observed for the two methods (FIG. 11B). The dual delivery efficiency into a single cell for the parallel method was 94.2%; while delivery efficiency was 82.3% when the transfection was performed in series (FIG. 11C). There was a clean 1:1 expression observed for co-delivery of mRNA in parallel with very few cells (1%) expressing only a single fluorescent reporter (FIG. 11B). In contrast, with in series transfections 3.3% of the population were single positive for GFP and 11.3% of the population were single positive for mCherry (FIG. 11C).

### Example 4—Multiple T Cell Donors

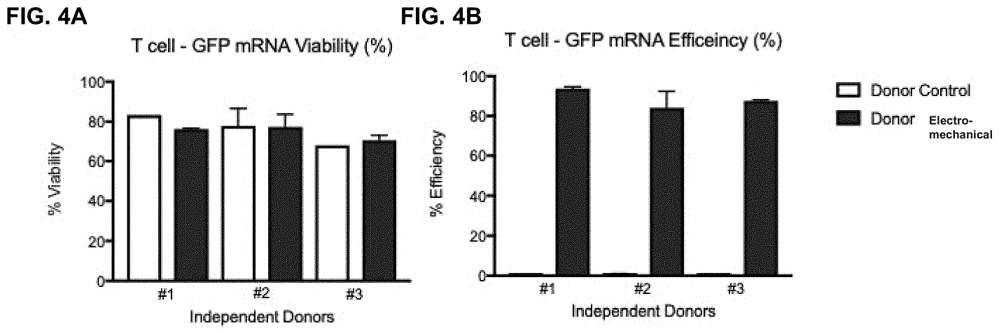

Donor heterogeneity is a constant in all cell therapy manufacturing and development pipelines, therefore it is vital that output metrics be assessed across multiple donors. For clinical manufacturing, source material including T cells from various donors can require re-characterization and comparability testing. It is therefore critical for cell therapy development to demonstrate that the results achieved during the above optimization effort translates to T cells sourced from a variety of starting material (FIGS. 4A-4B). Cells from three different healthy PBMC donors (demographics can be found in Table 1, below) were isolated, expanded, and then transfected with GFP mRNA with electro-mechanical transfection. All donors in this study met starting phenotypic and viability criteria outlined in the materials and methods section. This experiment demonstrated consistent results with multiple donor cellular material with less than 10% change in viability from no transfection controls (FIG. 4A). Additionally, the efficiency of GFP mRNA delivery had low variability (and exceeded 84%) for all three donors (FIG. 4B). Table 1—Expanded human T cells from three unique donors were transfected with a GFP reporter mRNA using electro-mechanical technology. This demographics table compares the three unique donors.

| | | | Weight | Height | | |
|---|---|---|---|---|---|---|
| Age | Sex | Race | (kg) | (cm) | Smoker | Donor |
| 43 | F | Hispanic | 93 | 173 | No | #1 |
| 55 | M | African American | 132 | 189 | Yes | #2 |
| 54 | M | African American | 124 | 182 | No | #3 |

### Example 5—Delivery of mRNA to Naïve Human T Cells

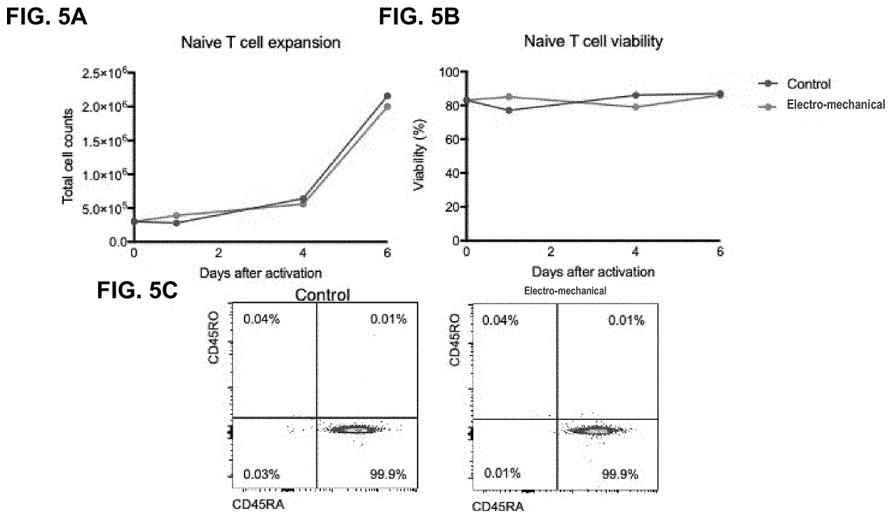

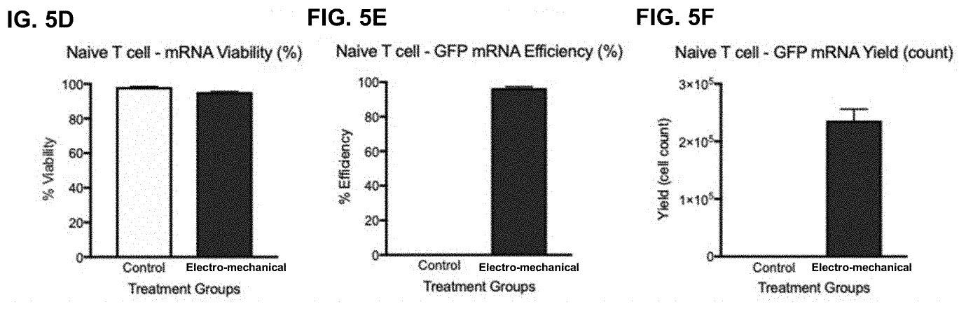

Although non-activated naïve T cells are of interest in cell engineering due to the relative simplicity in preparation and processing prior to transfection, this cell type is underrepresented in the cell and gene therapy field due to historical challenges associated with delivery of genetic information and the inability to perform retroviral transduction without first activating the T cells. In order to evaluate the performance of electro-mechanical transfection with this traditionally hard to transfect cell state, isolated naïve T cells (CD3⁺/CD4⁺/CD45RA⁺/CD45RO⁻) were transfected with mRNA encoding GFP (FIGS. 5A-5F). The naïve T cells were then expanded with soluble anti-CD3/anti-CD28 activation reagents and monitored for 6 days after transfection. The growth rates of these cells after transfection were equivalent to the non-processed control cells up to six days after activation (FIG. 5A) with no significant loss in viability (FIG. 5B). Additionally, the cells were stained for naïve T cell markers CD45RA and CD45RO (FIG. 5B), demonstrating there was no change in phenotype for the transfected cells and that the cells retained their naïve CD45RA⁺/CD45RO⁻ state. The viability of the transfected naïve T cells was equivalent to nontreated cells, at 95.4% and 98.3%, respectively (FIG. 5D). The delivery efficiency was observed at 96.7% (FIG. 5E), corresponding to a high total yield (FIG. 5F).

### Example 6—Manufacturing Volume Scale-up

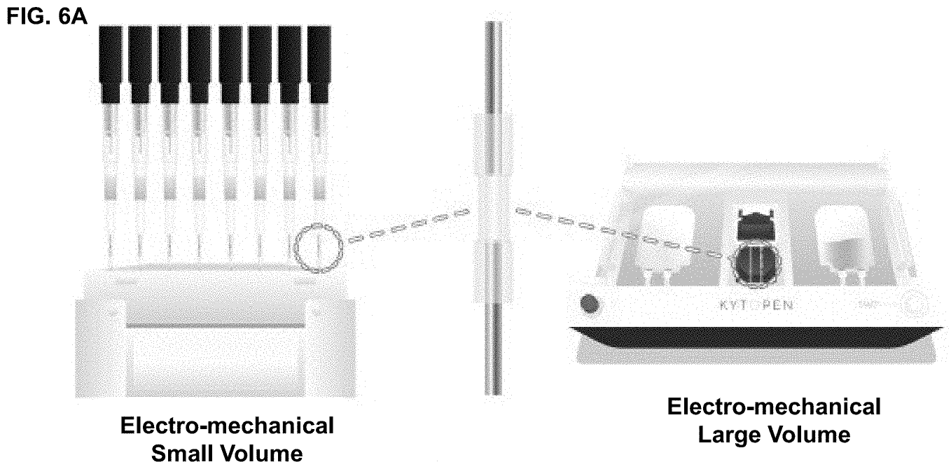

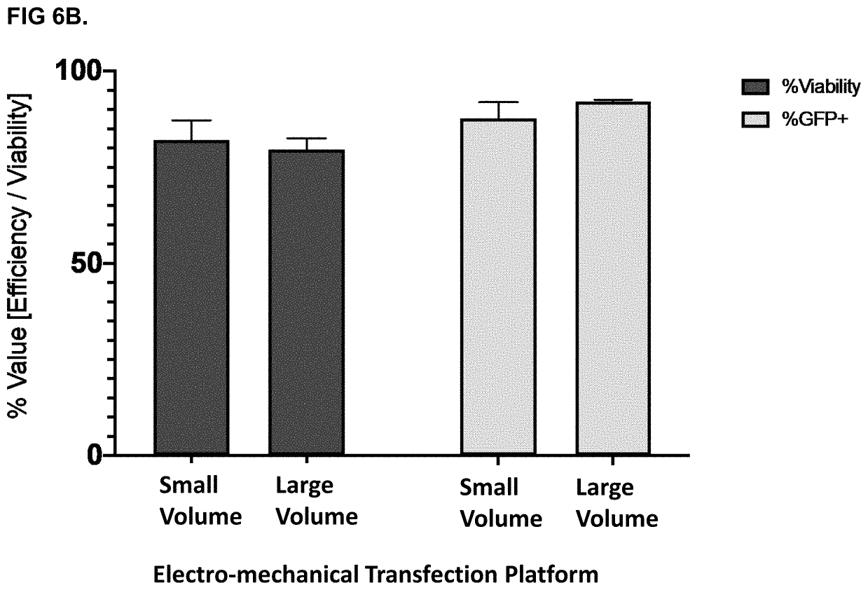

It has generally been accepted that standard electroporation requires additional optimization in the process of scaling up from research to manufacturing volumes, due to the changing geometries of both electrodes and cuvettes. To combat this issue, the field of electroporation-based transfection has seen the advent of numerous workarounds including the application of microfluidics, batch-based automation, and nanostructures. To date, these solutions have been unable to meet the need for both high-throughput development and large volume manufacturing requirements in the evolving cell and gene therapy industry. The electro-mechanical transfection technology has been configured into a large volume manufacturing platform (e.g., electro-mechanical systems and devices configured to be continuously resupplied with new cell suspension) utilizing the same flow cell previously described for small volume system (FIGS. 6A-6B). Due to its continuous flow nature, electro-mechanical transfection in the large volume platform scales with time. Therefore, processing larger volumes simply requires operating for a proportionally longer amount of time. The large volume electro-mechanical transfection solution is able to process up to 100 mL of fluid in roughly 2-3 minutes, transferring cell sample from an input to an output bag. Fluid flow is controlled via a peristaltic pump (Masterflex® L/S). The same parameters identified during optimization on the small-scale system of the invention are directly applied on the larger volume system because the electro-mechanical systems utilize the exact same transfection mechanism and components, regardless of scale (FIG. 6A). To demonstrate a 5 mL run (50-fold scale up from the data discussed above), we transfected 50M primary human T cells at a density of 10 x⁶/mL with 1 mg of mRNA (FIG. 6B). The results show no significant loss in cell viability 24 hours after electro-mechanical transfection, with viabilities of 73.5% and 71.0% via the small and large platforms, respectively (FIG. 6B). The observed delivery efficiency was also similar 24 hours after electro-mechanical transfection, 94.3% and 92.2% via the small and large platforms, respectively (FIG. 6B). Thus, electro-mechanical transfection can easily scale up for clinically relevant processing volumes.

### Example 7—Methods and Materials

The following methods and materials were used in gathering the data discussed in Examples 1-6.

### T Cell Culture and Expansion

Human peripheral blood cells (PBMCs) were purchased from STEMCELL Technologies™ (#70025). 100M PBMCs were thawed in 100 mL X-VIVO™ 10 media from Lonza (#04-380Q) with recombinant human IL-2 protein from R&D Systems™ (#202-IL). After a 24-hour culture out of thaw the PBMCs were activated with ImmunoCultTM human CD3/CD28 T cell activator reagent from STEMCELL Technologies™ (#10971) for 3 days according to the manufacturer's protocol. On day 4 the cells were pelleted (500 x g, 5 min) and transferred to a G-Rex100® from Wilson Wolf (#80500) with 500 mL fresh X-VIVO™ 10 media and recombinant human IL-2. Fresh recombinant human IL-2 was then added every 3 days for up to 12 total days in G-Rex® culture. Cells were then frozen into aliquots for future use with Bambanker™ cell freezing medium from Bulldog Bio (#BB01). Post expansion thawed aliquots of T cells were grown at 1 e6/mL density in RPMI 1640 media from Thermo Fisher (#11875119) with 10% fetal bovine serum (FBS) from Sigma-Aldrich® (#F-4135), penicillin-streptomycin solution from Corning® (#30-002-CI) and recombinant IL-2. Naïve primary human T cells (CD3⁺/CD4⁺/CD45RA⁺) were also sourced from STEMCELL Technologies™ (#70029) and cultured in RPMI with 10% FBS and IL-2 as described above. Cells were cultured at 37° C. with 5% CO₂ in a standard cell incubator. Cell viability and size were monitored during cell culture using CountessTM II (Thermo Fisher).

### Electro-mechanical Transfections

Transfections were performed with commercially sourced mRNAs encoding either GFP (#L7601) or mCherry (#L7203) from TriLink® Biotechnologies. T cells were counted, pelleted (500 x g, 5 min), and resuspended in a transfection buffer compatible with the invention at densities of 10-50×10⁶/mL. Payload was added at a fixed maximum of 10% volume and the cell:payload solution was mixed via pipetting.

Processing of cells with a small volume electro-mechanical device array were performed on a Perkin Elmer JANUS® G3 BioTx Pro Plus Workstation with an 8-tip Varispan™ head. The cell solutions were transferred to a 4° C. cooled mixing plate in a 96 well plate and aspirated through the tips of the electro-mechanical devices above the microfluidic channel. The solutions were then dispensed through the same tips at constant flow rates while specific electric fields were applied to these tips through an electric delivery manifold. The cells are delivered directly into cell culture media for recovery in a 96 deep well plate.

Processing of cells with the larger volume electro-mechanical transfection system was performed with a prototype in which fluid flows through an electro-mechanical transfection device placed between input and output bags connected by tubing, with fluid transfer controlled using a Masterflex® US peristaltic pump and PharMed® BPT tubing (L/S 13: #06508-13) from Cole-Palmer. The cell:payload solutions were transferred to an input vessel and then pumped through the channel at constant flow rates while specific electric fields were applied. The cells were then immediately transferred into cell culture media for recovery in an output vessel.

Final density in recovery solution for both platforms was 1×10⁶/mL, containing 10% transfection buffer and 90% cell culture media. Cells were cultured at 37° C. with 5% CO₂ in a standard cell incubator.

### Neon™ Transfections

The Thermo Fisher Neon™ transfection system was used according to manufacturer's instructions. Briefly, 5 M day 9 expanded T cells were resuspended in T buffer and loaded into the 100 μL Neon™ Pipette tip. The protocol ran was from the Neon™ T cell microporation protocol (2100V 1 pulse 20 ms). Processed cells were then transferred into a tissue culture vessel.

### 4D Nucleofector™ Transfections

The Lonza 4D Nucleofector™ system was used according to manufacturer's instructions. Briefly, 5 M day 9 expanded T cells were resuspended in freshly prepared human T cell nucleofection solution and were loaded into the 100 μL Lonza certified cuvette. The protocol ran was from the Amaxa™ 4D NucleofectorTM protocol for unstimulated human T cells (EO115). Processed cells were then transferred into a tissue culture vessel.

### Flow Cytometry Analysis

A Thermo Fisher Attune™ Nxt flow cytometer was used for assessment of viability and efficiency metrics. 200 μL of cultured cells were pelleted (500 x g, 5 min) and resuspended in Dulbecco's phosphate-buffered saline (DPBS) from Fisher Scientific (#14190250) with 7-AAD viability solution from eBiosciences™ (#00-6993-50). The cells were then analyzed on a volumetric read using the AttuneTM Nxt autosampler. Total cells were gated in the forward scatter (FSC) and side scatter (SSC) dot plots. Viable cells (7-AAD⁻) were then gated to determine delivery efficiency via expression of the fluorescent reporters. Total cell counts and yields were calculated from an applied dilution factor based on total volume of the cell culture (7.5X per 1 mL). Naïve T cell marker staining was performed with APC/Cy7 mouse anti-human CD45RA (#304128) and BV510 mouse anti-human CD45RO (#304232) antibodies from BioLegend®.

### Transcriptome Analysis

Whole cell pellets were collected from cell cultures at 6-hours and 24-hours post processing and stored at −80° C. All extraction of RNA, cDNA synthesis, next generation sequencing, and preliminary raw data normalized to controls was completed by GENEWIZ®. Normalized data was then analyzed (Excel—Microsoft) and graphed (Graph Pad—Prism 8) in house. Protein Analysis Through Evolutionary Relationships (PANTHER) classification system was used for gene ontology analysis.

### Other Embodiments

All publications, patents, and patent applications mentioned in this specification are herein incorporated by reference to the same extent as if each independent publication or patent application was specifically and individually indicated to be incorporated by reference. In particular, priority application U.S. Ser. No. 63/180,617 is incorporated by reference. In the event of a conflicting definition between this and any reference incorporated herein, the definition provided herein applies.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features hereinbefore set forth, and follows in the scope of the claims.

Other embodiments are within the claims.

1. A method of introducing a composition into a plurality of mammalian cells suspended in a flowing liquid, the method comprising:
(a) providing a device comprising:
i) an entry zone comprising a first inlet and a first outlet;
ii) first and second electrodes; and
iii) an active zone comprising a second inlet and second outlet; and
(b) selecting a combination of an electric field (E), an average flow velocity (u), a hydraulic diameter (d) in the active zone, a liquid conductivity (σ), liquid dynamic viscosity (μ), and liquid density (ρ) to give a dimensionless parameter π₅ having a value of between 1×10⁸ and 1×10¹⁰; wherein the dimensionless parameter π₅ is represented by:
$${Π}_{5} = \frac{ρσ {d}^{3}}{{μ}^{2}} ( \frac{{E}^{2}}{u} ) ,$$
and
(c) passing the plurality of cells and the composition through the active zone while providing the selected combination of (E), (u), (d), (σ), (μ), and (ρ), thereby introducing the composition into the plurality of mammalian cells.

2. The method of claim 1, wherein the composition is introduced into the plurality of cells at a flux of at least 1×10⁵ cells per minute per active zone.

3. The method of claim 1, wherein the entry zone and active zone are configured to provide an increase in average flow velocity.

4. The method of claim 1, wherein the composition is introduced into the plurality of mammalian cells without altering a desired cell surface marker.

5. A method of introducing a composition into a plurality of cells suspended in a flowing liquid, the method comprising:
(a) providing a device comprising:
i) an entry zone comprising a first inlet and a first outlet;
ii) first and second electrodes; and
iii) an active zone comprising a second inlet and second outlet and
(b) passing the plurality of cells and composition through the active zone while providing electrical energy from the first and second electrodes and providing mechanical energy at least partially from an average flow velocity; wherein the mechanical energy and the electrical energy together result in introducing the composition into the plurality of cells with an efficiency, a viability, and/or a yield at least equal to electroporation or mechanical poration alone with less electrical or mechanical energy than electroporation or mechanical poration require to achieve the efficiency, viability, and/or yield.

6. The method of claim 5, wherein the composition is introduced into the plurality of cells at a flux of at least 1×10⁵ cells per minute per active zone.

7. The method of claim 5, wherein the ratio of the electrical energy provided to the flowing liquid by the electric field to the mechanical energy provided by a pressure drop in the active zone is between 10³:1 and 10⁶:1.

8. The method of claim 5, wherein the entry zone and active zone are configured to provide an increase in average flow velocity.

9. A method of introducing a composition into a plurality of cells suspended in a flowing liquid, the method comprising:
(a) providing a device comprising:
i) an entry zone comprising a first inlet and a first outlet; and
ii) first and second electrodes;
iii) an active zone comprising a second inlet and second outlet, and having a hydraulic diameter (d); and
(b) providing a test portion of the plurality of cells and a test composition having together a liquid conductivity (a), liquid dynamic viscosity (p), and liquid density (p), and a ratio of cells to composition, and passing the test portion and test composition through the active zone at an average flow velocity (u) while applying an electric field (E), wherein at least one of (u), (E), (σ), (μ), and (ρ) is varied;
(c) identifying a range of a dimensionless parameter π₅ which includes a maximum cell viability, transfection efficiency, and/or engineered cell yield, for introduction of the test composition into the test portion of the plurality of cells, wherein
$${Π}_{5} = \frac{ρσ {d}^{3}}{{μ}^{2}} ( \frac{{E}^{2}}{u} ) ;$$
and
(d) passing the plurality of cells and the composition through the active zone with a combination of (u), (E), (σ), (μ), and (ρ) corresponding to a value of π₅ which includes at least one of the maximum cell viability, transfection efficiency, or engineered cell yield, thereby introducing the composition into the plurality of cells.

10. The method of claim 9, further comprising repeating step (b) with the test portion of cells and test composition having a second ratio of cells to composition, and/or the active zone having a second hydraulic diameter (d).

11. The method of claim 9, wherein the electric field (E) is held constant while varying the average velocity (u); or the average flow velocity (u) is held constant while varying the electric field (E).

12. The method of claim 9, wherein the composition is introduced into the plurality of cells at a flux of at least 1×10⁵ cells per second per active zone.

13. The method of claim 9, wherein the entry zone and active zone are configured to provide an increase in average flow velocity (u).

14. A method of introducing a composition into a plurality of human immune cells from a suspension collected from normal patient or donor blood and suspended in a flowing liquid, the method comprising:
(a) providing a device comprising:
i) an entry zone comprising a first inlet and a first outlet;
ii) first and second electrodes; and
iii) an active zone comprising a second inlet and second outlet; and
(b) selecting a combination of an electric field (E), an average flow velocity (u), a hydraulic diameter (d) in the active zone, a liquid conductivity (σ), liquid dynamic viscosity (μ), and liquid density (ρ) to give a dimensionless parameter π₅ having a value of between 1×10⁸ and 1×10¹⁰; wherein the dimensionless parameter π₅ is represented by:
$${Π}_{5} = \frac{ρσ {d}^{3}}{{μ}^{2}} ( \frac{{E}^{2}}{u} ) ,$$
and (c) passing the plurality of cells and the composition through the active zone while providing the selected combination of (E), (u), (d), (σ), (μ), and (ρ), thereby introducing the composition into the plurality of human immune cells to produce a therapeutic dose of transfected cells.

15. The method of claim 14, wherein the suspension is prepared using leukapheresis.

16. The method of claim 14, wherein the composition is introduced into the plurality of cells at a flux of at least 1×10⁵ cells per minute per active zone.

17. The method of claim 14, wherein the entry zone and active zone are configured to provide an increase in average flow velocity.

18. The method of claim 14, wherein the composition is introduced into the plurality of mammalian cells without altering a desired cell characteristic.
